(19) United States
(12) Patent Application Publication
(10) Pub. No.: US 2009/0269281 A1
(43) Pub. Date: Oct. 29, 2009

(54) Calcium/Calmodulin-Dependent Protein Kinase Kinase as a Control Point for Cardiac Hypertrophy

(75) Inventors: Stephen R. Grant, Fort Worth, TX (US); Thomas G. Valencia, Fort Worth, TX (US)

Correspondence Address: CHALKER FLORES, LLP, 2711 LBJ FRWY, Suite 1036, DALLAS, TX, 75234, US

(73) Assignee: University of North Texas Health Science Center at Fort Worth, Fort Worth, TX (US)

(21) Appl. No.: 12/207,177

(22) Filed: Sep. 9, 2008

Related U.S. Application Data
(60) Provisional application No. 60/972,779, filed on Sep. 15, 2007.

(51) Int. Cl. A61K 31/4745; A61P 21/00; A61K 49/00

(52) U.S. Cl. 424/9.2; 514/283

(57) ABSTRACT

The present invention includes compositions and methods for treating a patient with cardiac hypertrophy by providing the patient with an effective amount of a Calmodulin kinase kinase inhibitor that is sufficient to treat cardiac hypertrophy.

### CROSS-REFERENCE TO RELATED APPLICATIONS

This application claims priority to U.S. Provisional Application Ser. No. 60/972,779, filed Sep. 15, 2007, the entire contents of which are incorporated herein by reference.

### STATEMENT OF FEDERALLY FUNDED RESEARCH

This invention was made with U.S. Government support under Contract No. National Institutes of Health Grant No. R01-HL67152-01A1. The government has certain rights in this invention.

### TECHNICAL FIELD OF THE INVENTION

The present invention relates in general to the field of diagnosis and treatments for cardiac conditions, and more particularly, to compositions and methods for diagnosis and treatment of cardiac hypertrophy.

### INCORPORATION-BY-REFERENCE OF MATERIALS FILED ON COMPACT DISC

None.

### BACKGROUND OF THE INVENTION

Without limiting the scope of the invention, its background is described in connection with cardiac hypertrophy.

Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to dilated cardiomyopathy, heart failure, and sudden death. In the United States, approximately half a million individuals are diagnosed with heart failure each year, with a mortality rate approaching 50%.

Despite the diverse stimuli that lead to cardiac hypertrophy, there is a prototypical final molecular response of cardiomyocytes to hypertrophic signals that involves an increase in cell size and protein synthesis, enhanced sarcomeric organization, up-regulation of fetal cardiac genes, and induction of immediate-early genes, such as c-fos and c-myc. The causes and effects of cardiac hypertrophy have been extensively documented, but the underlying molecular mechanisms that couple hypertrophic signals initiated at the cell membrane to the reprogramming of cardiomyocyte gene expression remain poorly understood. Elucidation of these mechanisms is a central issue in cardiovascular biology and will be critical for designing new strategies for prevention or treatment of cardiac hypertrophy and heart failure.

Numerous studies have implicated intracellular Ca²⁺ as a signal for cardiac hypertrophy. In response to myocyte stretch or increased loads on working heart preparations, intracellular Ca²⁺ concentrations increase, consistent with a role of Ca²⁺ in coordinating physiologic responses with enhanced cardiac output. A variety of humoral factors, including angiotensin II (AngI), phenylephrine (PE), and endothelin-1 (ET-1), induce the hypertrophic response in cardiomyocytes and have the ability to elevate intracellular Ca²⁺ concentrations.

It is known that Gq-coupled receptor signaling in the heart that results in hypertrophy is thought to occur through a protein kinase C(PKC)-dependent mechanism. However, inhibition of PKC is insufficient to inhibit hypertrophic signaling. Activation of the MAP kinases p38 and ERK1/2 is thought to be the mechanism by which PKC induces hypertrophy.

Despite the development and availability of many methods for diagnosis and treatment of cardiac conditions, the morbidity and mortality related to cardiac hypertrophy remains very high.

### SUMMARY OF THE INVENTION

The present inventors have demonstrated that signaling through a newly-discovered Gq-coupled receptor (Urotensin II receptor (UIIR)) also induces hypertrophy through p38 and ERK1/2 activation. However, it was also found that PKC inhibition did not result in inactivation of p38 or ERK1/2 or reduce the hypertrophic phenotype. The inventors recognized that Gq-coupled receptors activate PLC which cleaves PIP2 into IP3 and DAG. IP3 causes the release of calcium from the sarcoplasmic reticulum of cardiomyocytes. The present invention is based on the finding that Calcium-dependent kinases (CaM kinases) activate the hypertrophic phenotype in cardiomyocytes. While a variety of second messenger cascades have been implicated in cardiac disease, the present inventors demonstrated for the first time that Gq-dependent signaling through the UIIR is dependent on CaM kinase kinase, which acts as a major control point for hypertrophy through UIIR and other Gq-coupled receptors.

More particularly, the present invention includes compositions, methods and kits for the diagnosis, detection, prevention and treatment of cardiac hypertrophy. A composition for treating a cardiac hypertrophy that includes an effective amount of a Calmodulin kinase kinase inhibitor sufficient to treat a patient suspected of having cardiac hypertrophy. In one embodiment the composition also includes a carrier, a diluent, a buffer, a second active agent, a dye, a salt and combinations thereof. In one aspect, the Calmodulin kinase kinase inhibitor comprises a cell-permeable naphthoyl fused benzimidazole compound. In another aspect, the Calmodulin kinase kinase inhibitor is a Calmodulin kinase kinase-specific inhibitor. In another aspect, the Calmodulin kinase kinase inhibitor is a Calmodulin kinase kinase-specific inhibitor comprises STO-609, derivatives and salts thereof. In another aspect, the Calmodulin kinase kinase inhibitor is provided profilactically to a patient having one or more predisposing factors for cardiac hypertrophy. In another aspect, cardiac hypertrophy comprises age-onset cardiomyopathy or angina. In another aspect, the Calmodulin kinase kinase comprises a CaM-KKα a CaM-KKβ and combinations thereof. The Calmodulin kinase kinase inhibitor may be a 7-H-Benz[de]benzimidazo[2,1-a]isoquinoline-7-one-3-carboxylic acid or salt thereof. In another aspect, the Calmodulin kinase kinase inhibitor is provided in a dose of at least 120 ng/ml.

In another embodiment, the Calmodulin kinase kinase inhibitor decreases the expression of Calmodulin kinase kinase selected from an aptamer, an siRNA, a cognate target antagonist or a peptide. In another aspect, the Calmodulin kinase kinase inhibitor comprises a nitric oxide synthase inhibitor. In another aspect, the Calmodulin kinase kinase inhibitor comprises a nitric oxide synthase inhibitor selected from (N-nitro-1-arginine [NNLA], or 7-nitroindazole sodium [7-NINA]). In another aspect, the Calmodulin kinase kinase inhibitor has the formula:

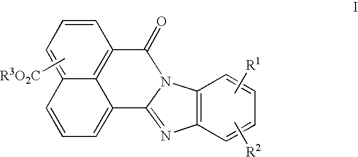

wherein R1, R2=H, halogen, alkyl, haloalkyl; R3=H, alkyl, substituted alkyl) or pharmaceutically acceptable salts thereof. In another aspect, the Calmodulin kinase kinase inhibitor is adapted for a dosage of between 0.001 to 500 gr/kg/day. In another aspect, the pharmaceutical composition is adapted for administration via parenteral, intravenous, oral, intramuscular, intraaortal, intrahepatic, intragastric, intranasal, intrapulmonary, intraperitoneal, subcutaneous, rectal, vaginal, intraosseal or dermal delivery. In another aspect, the pharmaceutical composition is in powder, tablet, gelatin, gelcap, capsule, soft-gel, chewable or liquid form. In another aspect, the composition may further comprise one or more vitamins, minerals, amino acids, lipids, nucleic acids, co-factors, pro-vitamins, and combinations of mixtures thereof.

Another embodiment of the present invention includes a pharmaceutical composition for treating a cardiac hypertrophy comprising a pharmaceutically effective amount of a Calmodulin kinase kinase inhibitor sufficient to treat a patient suspected of having cardiac hypertrophy. Another embodiment of the present invention is a composition for modulating a cardiac hypertrophy comprising an amount of a Calmodulin kinase kinase modulator sufficient to change the activity of the Calmodulin kinase kinase. In one aspect, the composition increases the amount of intracellular Calmodulin kinase kinase, intracellular Calmodulin kinase kinase mRNA, the stability of intracellular Calmodulin kinase kinase mRNA and combinations thereof. In one aspect, the composition decreases the amount of intracellular Calmodulin kinase kinase, intracellular Calmodulin kinase kinase mRNA, the stability of intracellular Calmodulin kinase kinase mRNA and combinations thereof. In one aspect, the composition increases the kinase activity of the Calmodulin kinase kinase. In one aspect, wherein the composition decreases the kinase activity of the Calmodulin kinase kinase mRNA.

Another embodiment of the present invention includes a method of treating a modulating muscle mass by administering to a patient in need thereof a composition comprising a Calmodulin kinase kinase inhibitor in a pharmaceutically acceptable carrier, in an amount insufficient to treat the cardiac hypertrophy. In one aspect, the muscle is cardiac muscle. Another embodiment of the present invention includes a method for treating or preventing hypertrophic cardiomyopathy in a mammal, the method by administering a Calmodulin kinase kinase inhibitor to the mammal, wherein the Calmodulin kinase kinase inhibitor is administered in an amount effective to treat or prevent heart failure in the mammal. In one aspect, the mammal is a human. In another aspect, the hypertrophic cardiomyopathy results from hypertension; ischemic heart disease; exposure to a cardiotoxic compound; myocarditis; thyroid disease; viral infection; gingivitis; drug abuse; alcohol abuse; periocarditis; atherosclerosis; vascular disease; hypertrophic cardiomyopathy; acute myocardial infarction; left ventricular systolic dysfunction; coronary bypass surgery; starvation; an eating disorder; or a genetic defect. In one aspect, the Calmodulin kinase kinase inhibitor is administered prior to, during, after the onset of cardiac hypertrophy. In one aspect, the Calmodulin kinase kinase inhibitor is administered prior to, during, after the diagnosis of heart failure in the mammal and/or administered prior to, during, after compensatory cardiac hypertrophy.

The present invention also includes a method of treating a disease in a mammal resulting from deficiencies of cardiac output by administering to the mammal a therapeutically effective amount of a pharmaceutical composition comprising a Calmodulin kinase kinase inhibitor. Another embodiment of the present invention is a method of modulating muscle mass by administering to a patient in need of an increase or a decrease in muscle mass an amount of a Calmodulin kinase kinase modulator sufficient to alter the muscle mass. In one aspect, the muscle is selected from cardiac and skeletal. In one aspect, the patient has muscle weakness and reduced pulmonary function, wherein the Calmodulin kinase kinase modulator increased muscle output. In one aspect, wherein the muscle weakness is caused by acute muscle usage. In yet another aspect, the muscle weakness is chronic.

The present invention also includes a method for diagnosing hypertrophic cardiomyopathy in a mammal, the method by measuring the Calmodulin kinase kinase activity from the mammal suspected of having the hypertrophic cardiomyopathy to the levels of kinase activity in a mammal known to have a normal cardiac function. Another method of the present invention includes screening compounds for the ability to prevent or treat the manifestations of heart failure, by contacting a Calmodulin kinase kinase to one or more candidate substances; and measuring the effect of the candidate substance on the kinase activity of the Calmodulin kinase kinase, wherein a candidate substance identified thereby is subsequently tested for improvement in the physiologic function of the heart of the mouse, thereby identifying a compound as therapeutic. In one aspect, a control for use with the method includes a Calmodulin kinase kinase that is a Val269 to Leu269 mutant, wherein known inhibitors of Calmodulin kinase kinase do not affect Calmodulin kinase kinase Val269 to Leu269 kinase activity. One example of a candidate substance is a naphthoyl fused benzimidazole compound and derivatives thereof. Yet another candidate substance includes derivative of STO-609. In operation, the candidate substance is compared to the known Calmodulin kinase kinase-specific inhibitor STO-609.

Yet another embodiment is a method for ameliorating the effects of physical exertion, the method comprising the administration to a person in need of such amelioration a composition of claim 1. In yet another embodiment, the present invention includes a diet for supporting a patient with cardiac hypertrophy comprising a nutritionally effective amount of a Calmodulin kinase kinase modulator to reduce the symptoms associated with cardiac hypertrophy and decreases muscle mass. The diet may further include essential fats of between about 0.1 to 10% total Kcal/day; carbohydrates restricted to about 0.1 to 10% total Kcal/day; and a protein content of between about 0.1 to 10% total Kcal/day of the diet.

Yet another embodiment of the present invention is a beverage concentrate that includes one or more carbohydrates, one or more electrolytes and one or more Calmodulin kinase kinase modulators in a concentration of between about 0.5% to about 5.0% weight percent. In one aspect, the beverage may be further defined as having the following ingredients:

| Ingredient | Approximate Concentration |
|---|---|
| Potassium | 2 meq/l |
| Sodium | 26 meq/l |
| Glucose | 4% |
| Pyruvate | 1% |
| a Calmodulin kinase kinase modulator | 0.1 to 10% |
| Emulsifier | 0.1 to 2.0% |
| water | balance. |

The present invention may be used in a food composition that includes a mixture of ingredients selected to make one or more snacks, soups, salads, cakes, cookies, crackers, breads, ice creams, yogurts, puddings, custards, baby foods, medicinal foods, sports bars, breakfast cereals and beverages and a Calmodulin kinase kinase inhibitor comprising a concentration of between about 0.1% to about 10.0% weight percent or 0.5 to 5.0% weight percent of the composition. The food composition may also include apple fiber, corn bran, soy fiber, pectin, guar gum, gum ghatti, and gum arabic, as well as mixtures thereof. The food composition may also include a binder material selected from the group consisting of rice flour, wheat flour, oat flour, corn flour, rye flour and potato flour, as well as mixtures thereof. The composition maybe formed into a pre-cooked edible and chewable product selected from the group consisting of breakfast cereals, snacks, soups, salads, cakes, cookies, crackers, puddings, ice creams, yoghurts, puddings, custards, baby foods, medicinal foods, sports bars, and beverages.

Yet another embodiment of the present invention includes a nutritional supplement that includes a nutritionally effective amount of a Calmodulin kinase kinase modulator sufficient to modulate muscle size.

The present invention also includes a transgenic mouse displaying manifestations of cardiac hypertrophy selected from the group consisting of: shortness of breath, angina, palpitations, lightheadedness, fatigue, syncope, dyspnea, elevated pressure in the left ventricle and left atrium and combinations thereof relative to a control mouse, wherein the genome of the mouse comprises a promoter operably linked to a nucleotide sequence encoding a Calmodulin kinase kinase, and a Calmodulin kinase kinase in its cardiac tissue at a level that is at least 3-fold higher than in cardiac tissue of a control mouse. In one aspect, the operable linked promoter is an inducible promoter, e.g., a cardiac-specific promoter. In another aspect, the gene promoter is a mouse or rat α-myosin heavy chain gene promoter. In another aspect, the Calmodulin kinase kinase is selected from a CaM-KKα a CaM-KKβ and combinations thereof. In one aspect, the Calmodulin kinase kinase is a Val269 to Leu269 mutant.

The present invention also includes a method for producing a transgenic mouse expressing a Calmodulin kinase kinase mRNA in cardiac tissue by introducing into an embryonal cell of a mouse a cardiac-specific gene promoter operably linked to a nucleotide sequence encoding a Calmodulin kinase kinase protein, wherein the promoter is capable of directing the expression of the nucleotide sequence encoding a Calmodulin kinase kinase protein in a cardiac-specific manner; transplanting the transgenic embryonal target cell formed thereby into a recipient female parent; and identifying at least one transgenic offspring containing the nucleotide sequence in the offspring's genome, wherein at from 8 to 18 months of age the offspring displays manifestations of cardiac hypertrophy selected from the group consisting of: shortness of breath, angina, palpitations, lightheadedness, fatigue, syncope, dyspnea, elevated pressure in the left ventricle and left atrium and combinations thereof relative to a control mouse, and expresses Calmodulin kinase kinase protein mRNA in its cardiac tissue at a level which is at least 3-fold higher than in cardiac tissue of a control offspring. In one aspect, the offspring is further characterized by not overexpressing the Calmodulin kinase kinase protein mRNA in skeletal muscle. In one aspect, the Calmodulin kinase kinase protein is selected from the group consisting of CaM-KKα a CaM-KKβ and combinations thereof.

The present invention also includes a method for screening compounds for the ability to prevent or treat the manifestations of heart failure in a mouse by providing a transgenic mouse from 8 to 18 months of age displaying manifestations of cardiac hypertrophy selected from the group consisting of: shortness of breath, angina, palpitations, lightheadedness, fatigue, syncope, dyspnea, elevated pressure in the left ventricle and left atrium and combinations thereof relative to a control mouse, wherein the genome of the mouse comprises an α-myosin heavy chain gene promoter operably linked to a nucleotide sequence encoding Calmodulin kinase kinase mRNA, and expresses Calmodulin kinase kinase mRNA in its cardiac tissue at a level which is at least 3-fold higher than in cardiac tissue of a control mouse; administering a compound to the mouse; and measuring an improvement in the physiologic function of the heart of the mouse and thereby identifying a compound as therapeutic.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B. Hypertrophic agonists stimulate the expression of UIIR in cardiomyocytes. FIG. 1A. Cardiomyocytes were cultured in medium containing 0.2% FBS and either PE (10 μM) or AngII (100 nM) for 48 hours. Total RNA was isolated and reverse transcribed. cDNA was used as template to amplify UIIR. FIG. 1B. Cardiomyocytes were cultured in medium containing 0.2% FBS and either PE (10 μM) or AngII (100 nM) for 48 hours. Total protein was isolated and UIIR detected by western blot.

FIGS. 2A to 2B. UII stimulation of hypertrophy marker genes requires CaMKK. FIG. 2A. Cardiomyocytes were cultured in medium containing 0.2% FBS and transfected with either ANF (50 ng/well), SkA (50 ng/well) or MEF2 (250 ng/well) reporters. Three hours post-transfection, cells were re-fed in media supplemented with 0.2% FBS. Following transfection, designated culture wells were infected with AdUIIR as described. Designated wells were then pre-treated with STO-609 (250 ng/mL) 1 h prior to stimulation with UII (100 nM). Luciferase activity was determined by luminometry (**, p<0.01; ***, p<0.001; as indicated). FIG. 2B. Cardiomyocytes were cultured in medium containing 0.2% FBS and infected with AdUIIR. Cells were stimulated with UII (100 nM) in the absence or presence of STO-609 (250 ng/mL) for 48 h. The relative expression of ANF, BNP, bMHC, SkA and GAPDH was analyzed by semi-quantitative PCR using gene-specific primers.

FIGS. 3A to 3C. CaMKI is specifically activated by UII stimulation. FIG. 3A. left panel: Cardiomyocytes were cultured and infected with AdUIIR as described. Cells were stimulated with UII (100 nM) for various times up to 60 min. Whole cell lysates were subjected to Western blotting to determine the relative change in phosphorylation of CaMKI. FIG. 3A right panel: Cardiomyocytes were cultured as above and stimulated with UII in the absence or presence of STO-069 for 2 and 5 min. Whole cell lysates were subjected to Western blotting to determine the relative change in phosphorylation of CaMKI. FIG. 3B. Cardiomyocytes were cultured and transfected with ANF, SkA or MEF2 as well as CaMKI and then infected with AdUIIR as described. Cells were treated with STO-609 (250 ng/mL) 1 h prior to stimulation with UII (100 nM)). Luciferase activity was determined by luminometry (**, p<0.01; ***, p<0.001; compared with control; ††\, p<0.001, compared with UII stimulation alone). FIG. 3C. Cardiomyocytes were cultured and transfected with ANF, SkA or MEF2 and infected with AdUIIR as described. Cells were treated with AKTi (5 μM) 1 h prior to stimulation with UII (100 nM). Luciferase activity was determined by luminometry (*, p<0.05; ***, p<0.001; n.s.=not significant; as indicated).

FIGS. 4A to 4D. MAPKs are activated by UII, require CaMKK, and are required for stimulation of ANF, SkA and MEF2 by UII or CaMKI. FIG. 4A. Cardiomyocytes were cultured and infected with AdUIIR as described. Cells were stimulated with UII (100 nM) for various times up to 60 min. Whole cell lysates were subjected to Western blotting to determine the relative change in phosphorylation of p38 and ERK1/2. FIG. 4B. Cardiomyocytes were cultured as above and stimulated with UII in the absence or presence of STO-069 for 2 and 5 min. Whole cell lysates were subjected to Western blotting to determine the relative change in phosphorylation of p38 and ERK1/2. FIG. 4C. Cardiomyocytes were cultured in medium containing 0.2% FBS and transfected with either ANF (50 ng/well), SkA (50 ng/well) or MEF2 (250 ng/well) reporters. Three hours post-transfection, cells were re-fed in media supplemented with 0.2% FBS. Following transfection, designated culture wells were infected with AdUIIR as described. Designated wells were then pre-treated with either STO-609 (250 ng/mL), SB203580 (10 μM) or U0126 (10 μM) 1 h prior to stimulation with UII (100 nM). Luciferase activity was determined by luminometry (***, p<0.001; compared with control; †††, p<0.001; compared with UII stimulation; n.s.=not significant). FIG. 4D. Cardiomyocytes were cultured and transfected with ANF, SkA or MEF2 as well as CaMKI as described in the absence or presence of SB203580 (10 μM) or U0126 (10 μM). Luciferase activity was determined by luminometry (**, p<0.01 and ***, p<0.001; compared with control; ††, p<0.01 and †††, p<0.001; compared with UII stimulation; n.s.=not significant)

FIGS. 5A and 5B. Dominant-negative p38 inhibits UII and CaMKI stimulation of MEF2. FIG. 5A. Cardiomyocytes were cultured and co-tranfected with MEF2 reporter and dominant-negative p38 and infected with AdUIIR as described. Luciferase activity was determined by luminometry (**, p<0.01; ***p<0.001; as indicated). FIG. 5B. Cardiomyocytes were co-transfected with MEF2 reporter, CaMKI and dominant-negative p38. Luciferase activity was measured by luminometry (***, p<0.001; as indicated).

FIGS. 6A to 6D. CaMKK is required for UII-dependent HDAC5/14-3-3b association and activation of PKD. FIG. 6A. Cardiomyocytes were cultured and infected with AdUIIR as described. Cells were stimulated with UII (100 nM) for various times up to 60 min. Whole cell lysates were subjected to immunoprecipitation of 14-3-3$ and western blotted to determine the relative change in abundance coimmunoprecipitated HDAC5. FIG. 6B. Cardiomyocytes were cultured as above and stimulated with UII in the absence or presence of STO-069, SB203580 and U0126 for 60 min. Whole cell lysates were subjected to Western blotting to determine the relative change in coimmunoprecipitated HDAC5. FIG. 6C. Cardiomyocytes were cultured and infected with AdUIIR as described. Cells were stimulated with UII (100 nM) for various times up to 60 min. Whole cell lysates were subjected to Western blotting to determine the relative change in phosphorylation of PKD. FIG. 6D. Cardiomyocytes were cultured as above and stimulated with UII in the absence or presence of STO-069 for 2 and 5 min. Whole cell lysates were subjected to Western blotting to determine the relative change in phosphorylation of PKD.

FIGS. 7A and B. CaMKK is required for AngII and Et-1 activation of ERK1/2 and PKD. Cardiomyocyte were cultured as described and stimulated with AngII (100 nM) or Et-1 (10 nM) for 10 min in the absence or presence of STO-609 (250 ng/mL). Whole cell lysates were subjected to Western blotting to determine the relative change in phosphorylation of ERK1/2 (FIG. 7A) or PKD (FIG. 7B).

FIG. 8 is a schematic overview of the role of CaMK signaling in UII stimulation of hypertophy genes. The novel crosstalk between the CaMKs and the MAPKs is demonstrated herein.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention is based on the finding that CaM kinase kinase inhibition results in the loss of Gq stimulated hypertrophy signaling through the Urotensin II receptor, with preliminary data suggesting the same for AngIIR and Et-1R. Furthermore, the CaM kinase kinase pathway cross-talks with the MAP kinases p38 and ERK1/2 (probably through CaM kinase I) as well as protein kinase D activity, which is also dependent on CaM kinase kinase through Urotensin, Angiotensin II and Endothelin-1 receptor activation.

The discovery that CaM kinase kinase integrates signaling through Gq-coupled receptors in the heart may provide a central control mechanism for reducing the hypertrophic response under conditions where Gq signaling is active (i.e., mechanical stress to myocardium). Existing technologies for the treatment of heart disease (namely reduction of hemodynamic load by ACE inhibitors or beta blockers) do not address the hypertrophic phenotype of the cardiomyocyte. We believe that the combined inhibition of CaM kinase kinase along with therapies that reduce hemodynamic load would be beneficial to maintain circulatory homeostasis and reduce cardiac compensation in the form of hypertrophy. This invention would overcome the limitations of current therapies by targeting the inhibition of the hypertrophic phenotype in the heart. Historically, the CaM kinase kinase cascade has been overlooked as regards the hypertrophic phenotype. These results demonstrate that CaM kinase kinase may be central to the control of Gq-induced cardiac hypertrophy.

While prior work has focused mainly on the ability of PKC to transduce Gq-signaling to the MAP kinases resulting in hypertrophy, a need remains for compositions and methods to treat cardiac hypertrophy. A need remains because inhibition of PKC does not lead to the inhibition of the MAP kinases; or, PKC inhibition does not account for the entire activity of the MAP kinases leading some to suggest other pathway involvement.

The present invention demonstrates that CaM kinase kinase (CaMKK) is involved critically in Gq-receptor signaling and cross-talk with the MAP kinases. The present invention demonstrates, for the first time, that dysregulation of CaMKK in cardiomyocytes results in cardiac hypertension. Furthermore, the present invention includes compositions and methods for targeting CaMKK to reduce cardiac hypertrophy in a well-known in vitro model system for cardiac hypertrophy. The present invention may be used alone or in conjunction with present therapies to relieve cardiac stress focus. Present therapies rely almost exclusively on normalizing hemodynamic load through inhibition of systemic vasoconstriction.

It was found that CaM kinase kinase inhibition can be combined with existing therapies to lessen the overall hemodynamic stress on the heart thereby normalizing systemic pressure and at the same time, reducing the hypertrophic phenotype. Since some preexisting therapies reduce hemodynamic load through inhibition of AngII signaling, it is possible that CaM kinase kinase inhibition itself will result in inhibition of AngII-induced vasoconstriction. Therefore, inhibition of CaM kinase kinase can be used to reduce the amount of pharmaceutics that the patient would have to take to normalize hemodynamic stress on the heart.

The transgenic animals of the present invention include those that have a substantially decreased probability of spontaneously developing cardiac hypertrophy, and those which have a substantially increased probability of spontaneously developing cardiac hypertrophy, when compared with non-transgenic littermates. As used herein, the terms “substantially increased” or a “substantially decreased” probability of spontaneously developing cardiac hypertrophy refer to a statistically significant increase or decrease, respectively, of measurable symptoms of cardiac hypertrophy is observed when comparing the transgenic animal with a non-transgenic littermate(s).

To understand how the signaling mechanism Urotensin II (UII) induces hypertrophy, primary rat cardiomyocytes were infected with an adenoviral vector that expressed the UII receptor. Using a combination of UII stimulation and pharmacological inhibitors, we determined that UII stimulation of hypertrophic genes, p38 and ERK1/2 MAP kinase activation as well as the activation of MEF2 required CaM kinase kinase (CaMKK). CaMKI was activated by UII which was inhibited by STO-609. Constitutively-active CaMKI was able to rescue UII stimulation of ANF and skeletal actin (SkA) in the presence of STO-609. In a mechanism not previously described in cardiomyocytes, UII stimulation of p38 and ERK1/2 required CaMKK. In addition, activation of ANF, SkA and MEF2 by UII or CaMKI required p38 and ERK1/2. UII stimulated the association of histone deacetylase 5 (HDAC5) with 14-3-3β which was blocked by inhibition of CaMKK or ERK1/2, but not p38. It is also demonstrated herein that UII-dependent activation of PKD required CaMKK. Taken together, these results identify components of an important intracellular signaling pathway through which UII activates CaMKK to promote hypertrophy of cardiomyocytes.

In the adult myocardium, cardiac hypertrophy results from various forms of physical stress (chronic hypertension, severe mechanical load, volume overload, myocardial stress from disease or stress from infarction or coronary insufficiency). On the cardiomyocyte level, the hallmarks of hypertrophy are an increase in cell size, increased protein synthesis, and sarcomeric reorganization (1, 2). The hypertrophic changes in cardiomyocyte phenotype are preceded by the re-expression of a fetal gene program in the left ventricle. Most notably, there is a shift in the expression from the adult β-myosin heavy chain (βMHC) to fetal b-myosin heavy chain (αMHC) and increased expression of skeletal α-actin (SkA), atrial natriuretic factor (ANF) and brain natriuretic peptide (BNP)(3-6). The switch from the adult gene expression pattern to the fetal pattern involves both the calcium/calmodulin-dependent protein kinases (CaMKs) as well as the mitogen activated protein kinases (MAPKs) (7-10). Interestingly, a crosstalk mechanism between CaMKs and MAPKs has been demonstrated in neurons (11). To date, no such mechanism has been demonstrated in cardiomyocytes.

An important point of convergence for hypertrophy induction by CaMKs and MAPKs is the myocyte enhancer factor 2 (MEF2)(12, 13). In the normal heart, MEF2 is held inactive by class II histone deacetylases (HDACs). In order for MEF2 to be active, HDACs must be phosphorylated and removed from the nucleus via the chaperone 14-3-3β (14-17). In addition, MEF2 itself must be phosphorylated by p38 MAPK to be fully active (18). CaMKI does not have access to the nucleus. This led us as well as others to speculate that there must be an effector kinase downstream of CaMKI that has access to the nucleus and can phosphorylate HDAC4/5. To date, there has been no kinase discovered downstream of CaMKI to provide this function. Recently; however, it was demonstrated that PKD phosphoryates HDAC4/5 leading to their nuclear export and activation of MEF2 in cardiomyocytes (19).

G-protein coupled receptors (GPCRs) that couple to Gq, such as the angiotensin II receptor (AngIIR) and the endothelin-1 receptor (ET-1R) are capable of activating the CaMK and MAPK cascades and are involved in cardiac hypertrophy (20-23). Mechanical stress has been shown to result in the release of both AngI and ET-1 from the heart leading to an autocrine stimulation of myocytes (24-29). Moreover, AngII stimulates the expression of Endothelin-1B receptor in cardiomyocytes (30). More important, signaling through Gq-coupled receptors has been implicated in the development of compensated cardiac hypertrophy and ultimately, decompensated hypertrophy that leads to failure. Indeed, transgenic models of Gq activation have shown that moderate degrees of Gq signaling stimulate adaptive hypertrophy (31-33), whereas high degrees of Gq signaling result in maladaptive cardiomyocyte apoptosis (34-37). The recently discovered UIIR is also Gq-coupled, its expression in cardiomyocytes increases during hypertrophy (38), and its ligand (UII) is also expressed in cardiomyocytes (39).

The Urotensin II receptor (UIIR) is a recently de-orphanized GPCR that is coupled to Gq and is activated by its peptide ligand, Urotensin II (UII) (40). UII and its receptor are expressed in the healthy adult heart at low levels and become overexpressed under pathological conditions that lead to hypertrophy (38, 41-43). By itself, UII is capable of inducing the hypertrophic phenotype in cultured cardiomyocytes only when sufficient receptor is expressed (44). Using adenoviral up-regulation of UIIR in cardiomyocytes, Onan, et al., showed that UII induced the hypertrophic phenotype as evidenced in enlargement of cardiomyocytes, sarcomeric reorganization as well as activating ERK 1/2 and p38 MAP kinases (44). Interestingly, MAP kinase activation and hypertrophy was shown to be independent of PKC activity. Since UII stimulation results in increased [Ca²⁺]ᵢ, it is likely that the CaMK cascade is active under these conditions. It is demonstrated herein that the hypertrophic up-regulation of UIIR and the resultant increase in Gq signaling and intracellular calcium activate CaMKK resulting in the downstream activation of CaMKI. CaMKI, once active, regulates the activities of ERK1/2 and p38, which can account for the PKC-independent hypertrophic phenotype observed by Onan, et al.

UIIR is a newly discovered Gq coupled receptor and many questions still remain concerning UIIR expression during cardiac disease states as well as its role in downstream signaling mechanisms that contribute to the hypertrophic phenotype. The present inventors demonstrate herein that Gq-coupled receptor agonists phenylephrine (PE) and AngII upregulate UIIR in cardiomyocytes. The inventors also demonstrate that UII induced activation of ANF, SkA and MEF2 in cultured cardiomyocytes was dependent on CaMKK as was activation of p38 and ERK1/2 MAPKs. It was also found that UII activated CaMKI and both UII and CaMKI dependent activation of ANF, SkA and MEF2 required p38 and ERK1/2 MAPKs. UII-stimulated HDAC5 association with MEF2 also required CaMKK as well as ERK1/2. Finally, it was found that UII can activate protein kinase D in a CaMKK-dependent manner. These data, taken together, demonstrate a central role for CaMKK in UII stimulation of hypertrophy and demonstrate a novel crosstalk mechanism between the CaMKs and the MAPKs not previously described in cardiomyocytes.

Cell Culture and Treatments. Cardiomyocytes were isolated from 2-4 day old Sprague-Dawley rats using the neonatal cardiomyocyte isolation system (Worthington Biolabs) based on a previously described protocol (45). Cells were plated in medium 199 supplemented with 10% fetal bovine serum (FBS, Atlanta Biologicals) and gentamycin (Fisher). Cells were maintained in medium 199 supplemented with 1.0% FBS and gentamycin. Prior to any treatment, cardiomyocyte cultures were incubated in medium 199 containing 0.2% serum for 24 hours. For stimulation with UII (100 nM unless indicated otherwise), cardiomyocyte cultures were first infected with AdUIIR as described previously (44). Cells were incubated in medium 199 containing 0.2% FBS prior to stimulation with the indicated reagents for reporter assays, RT-PCR, western blotting and immunoprecipitation. Cells were pre-treated with the inhibitors STO-609 (250 ng/mL; Sigma), SB203580 (10 μM; Sigma), U0126 (10μ″; Sigma) or 1L-6-Hydroxymethyl-chiro-inositol 2-(R)-2-O-methyl-3-O-octadecylcarbonate (AKTi; 5μ″; EMD Biosciences) for 60 min prior to stimulation as indicated. Plasmids and Adenoviral Constructs. In order to quantify hypertrophy-sensitive promoter activation, two luciferase-based promoter-reporter plasmid constructs were used: (i) A 700 bp fragment (NP337) of the ANF promoter (received from Mona Nemer, University of Montreal, Quebec, Canada) which was described previously (46); and (ii) a 400 bp fragment of the SkA promoter (received from Robert J Schwartz and Michael D. Schneider, Baylor College of Medicine, Houston, Tex.), also described previously (47). In order to quantify the activation of MEF2, we used a luciferase-based MEF2 enhancer-reporter plasmid that contained three MADS box repeats immediately upstream from a minimal promoter and luciferase structural gene (received from Eric Olson, University of Texas Southwestern Medical Center, Dallas, Tex.) and was previously described (48). The plasmid vector encoding CaMKI was obtained from Eric Olson (University of Texas Southwestern Medical Center, Dallas, Tex.)(49). The plasmid vector encoding dominant negative p38 (p38AF) was obtained from J. Han (Scripps Research Institute, La Jolla, Calif.) and were previously described (50-52). The adenoviral vectors that express UIIR were received from Walter Thomas (Baker Heart Institute, Melbourne, Australia) and were described previously (42, 44).

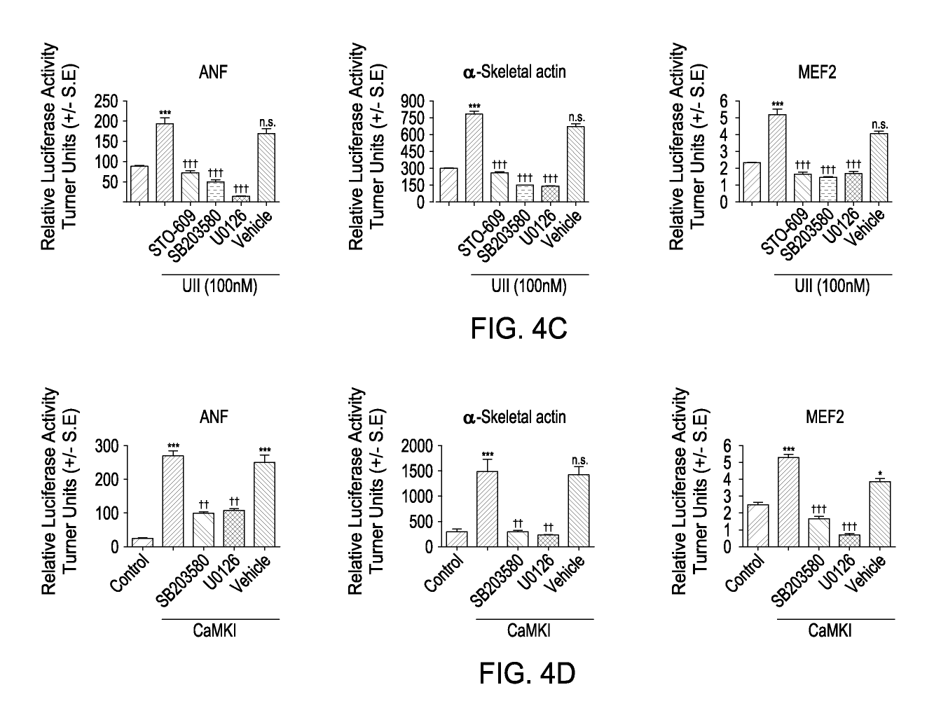

Transient Transfection and Luciferase Assay. For transfections, cardiomyocytes were cultured in 12-well tissue culture plates as described. Once cultures were incubated in medium 199 with 0.2% serum for 24 hours, cultures were either infected with AdUIIR as described or were just transfected. FIG. 5 describes the overall scheme of infection and/or infection. Depending on the study, cardiomyocytes were transfected with either control empty vector (pSG5), ANF promoter-reporter (50 ng/well), SkA promoter-reporter (50 ng/well), MEF2 enhancer-reporter (250 ng/well), CaMKI (10 ng/well) or p38aAF (100 ng/well) using LipofectAMINE# Plus reagent (Invitrogen, Carlsbad, Calif.) per manufacturer's protocol. Three hours after transfection, the cultures were washed and re-fed with medium 199 and 0.2% FBS. Depending on study, 24 hours post transfection, cells were stimulated with UII (concentrations as indicated); cultures that were to receive pharmacological inhibitors were incubated with them 45 minutes prior to UII stimulation at the indicated doses. Cardiomyocyte cultures were harvested 24-72 hours post treatment (per sample) and luciferase activity was determined by luminometry (Model TD 20/20 Luminometer, Turner Designs, Sunnyvale, Calif.) using a commercially available kit (Luciferase Substrate, Promega, Madison, Wis.).

Reverse Transcription and Semi-quantitative PCR—Cardiomyocytes were cultured in 100-mm dishes at a density of 1×10⁵ cells/cm². 24 h post-plating, cells were treated as indicated. Following treatment, total RNA was isolated with TRIzol® reagent (Invitrogen) cDNA was synthesized using Super-Script™III (Invitrogen) per the manufacturer's instructions. Semi-quantitative PCR was then performed using gene-specific primers for UIIR, hypertrophy marker genes and glyceraldehyde-3-phosphate dehydrogenase (GAPDH) (Integrated DNA Technologies). Sequences of gene-specific primers were as follows: for UIIR, sense 5′-CTGTGACTGAGCTGCCTGGTGAC-3′ (SEQ ID NO: 1) and antisense 5′-GGTGGCTATGATGAAGGGAAT-3′ (SEQ ID NO: 2); for ANF, sense 5′-TGCCGGTAGAAGATGAGGTC-3′ (SEQ ID NO: 3) and antisense 5′-AGCCCTCAGTTTGCTTTTCA-3′ (SEQ ID NO: 4); for BNP sense, 5′-GACGGGCTGAGGTTGTTTTA-3′ (SEQ ID NO: 5) and anti-sense 5′-TTGTGCTGGAAGATAAGAAA-3′ (SEQ ID NO: 6); for αMHC sense, 5′-CTCCCAAGGAGAGACGACTG-3′ (SEQ ID NO: 7) and antisense 5′-CCCTTGGTGACGTACTCGTT-3′ (SEQ ID NO: 8); for SkA sense, 5′-TGCCCATTTATGAGGGCTAC-3′ (SEQ ID NO: 9) and antisense 5′-GGCATACAGGTCCTTCCTGA-3′ (SEQ ID NO: 10); and for GAPDH sense, 5′-GTGTGAACGGATTTGGCCGTATGG-3′ (SEQ ID NO: 11) and antisense 5′-TCATACTTGGCAGGTTTCTCCAGG-3′ (SEQ ID NO: 12).

Western Blot Analysis and Immunoprecipitations. Cardiomyocytes were cultured in 100-mm dishes as described in the previous section. 24 h post-plating, cells were treated with UII and various pharmacological inhibitors as indicated. Following treatment, cells were lysed, and the relative abundance of UIIR, active p38 and ERK1/2, and active PKD was determined with commercially-available antibodies (Alpha Diagnostic, and Cell Signaling Technologies, Inc., respectively). The antibody for CaMKI was obtained from T. Soderling (Vollum Institute, Oregon Health Sciences University, Portland Oreg.).

For immunoprecipitation studies, Cardiomyocytes were cultured in 100-mm as described in the previous section. 24 h post-plating, cells were treated with UII and various pharmacological inhibitors as indictated. Following treatment, 14-3-3β was immunoprecipitated from whole cell lysate using a commercially available antibody (Abcam) and a protein G immunoprecipitation kit (IP50 Kit, Sigma, following manufacturer's protocol). HDAC5 was detected using a commercially-available antibody (Cell Signaling Technologies, Inc.).

Statistical Analysis. All results are expressed as the mean±S.E. Data were analyzed with GraphPad Prism software (version 4.0, GraphPad Software Inc.) using either student's T-test or one-way analysis of variance and Bonferroni's post-hoc test for inter-group comparisons. p values <0.05 were considered statistically significant.

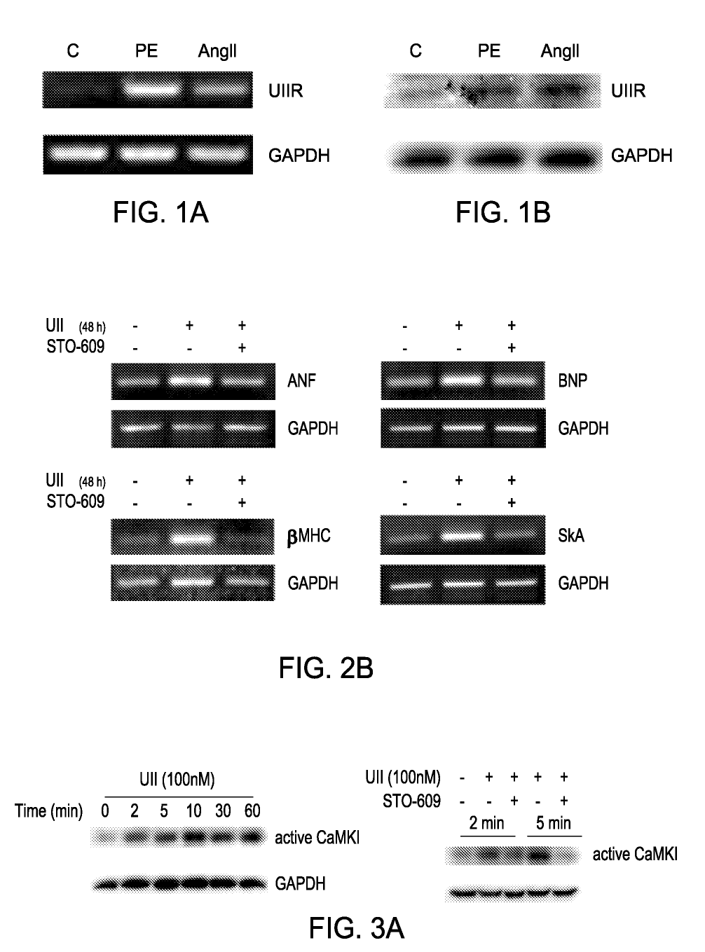

Expression of UIIR in myocytes treated with hypertrophic agonists. In order to validate the array data, we designed PCR primers to amplify UIIR. Cardiomyocyte cultures were treated with 10 μM PE or 100 nM AngII for 24 hours and total RNA was extracted following the Trizol protocol (Invitrogen). Total RNA was then used for template for reverse transcription. The cDNA generated from the RT reaction was then used as template to amplify UIIR and GAPDH as control. Treatment of cardiomyocytes with both PE and AngII for 24 hours up-regulated the UIIR messenger RNA (FIG. 1A). To our knowledge, this is the first evidence that demonstrates the ability of an α-1 adrenoreceptor agonist up-regulating UIIR in cardiomyocytes.

Next, the inventors determined whether increased mRNA of UIIR correlated with an increase in UIIR protein. To this end, we stimulated cardiomyocyte cultures with AngII and PE as before and isolated total protein. The total protein was resolved by PAGE (12% acrylamide), transferred to nitrocellulose, and blotted for UIIR using a specific antibody.

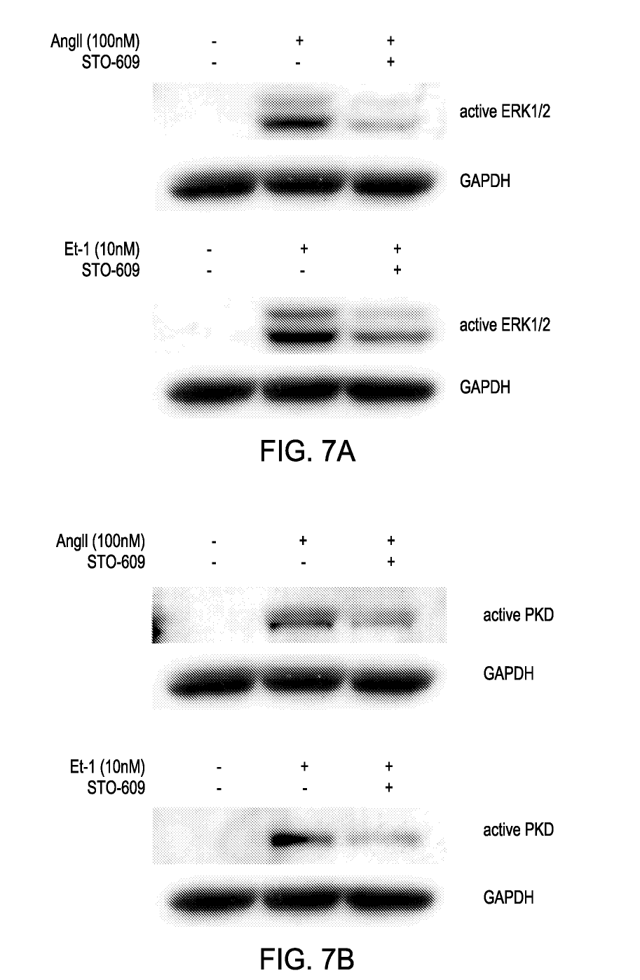

Treatment of cardiomyocytes with both PE and AngII resulted in the up-regulation of UIIR with no change in the expression of GAPDH (FIG. 7). These data demonstrate that along with an increase in mRNA, the protein for UIIR is up-regulated in cardiomyocytes in response to PE and AngII.

UII stimulates ANF, SkA and MEF2 promoter/enhancer activities. To determine the dose-dependency of UII stimulation of ANF, SkA and MEF2 promoter reporters was evaluated. UII was able to increase both ANF and SkA and MEF2 promoter-reporter activities in a dose-dependent manner (data not shown). In order to determine whether UII stimulation of ANF, SkA and MEF2 was specific for cultures infected with AdUIIR, primary rat cardiomyocytes were transfected as described. Promoter reporter activities were significantly increased in AdUIIR-GFP infected cardiomyocytes when compared with AdGO-GFP infection (ANF: t=10.04; p<0.0001; SkA: t=3.858, p=0.0182; MEF2:)(data not shown).

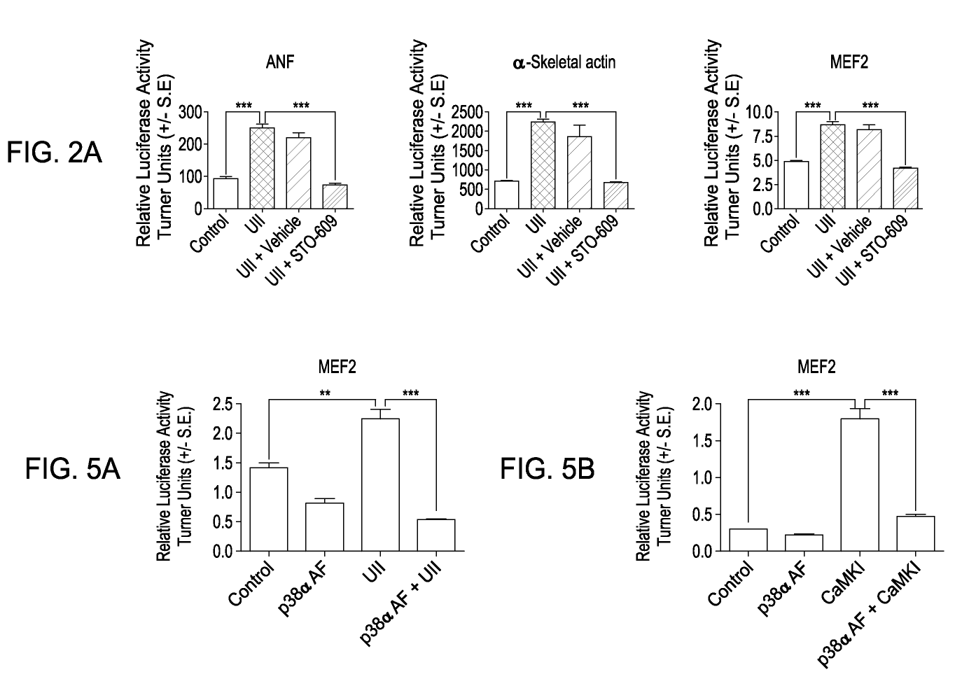

Specific CaMKK inhibition abolishes UII stimulation of ANF, SkA and MEF2 reporter activities. A role for UII in hypertrophy has been demonstrated in cultured cardiomyocytes; however, the complete signaling mechanism through which this is accomplished has not been determined. Since UII signals through a Gq-coupled receptor, we hypothesized that the resulting rise in cytosolic calcium could activate CaMKK leading to hypertrophic gene induction. To test whether UII stimulation of hypertrophy-sensitive promoter activity was dependent on CaMKK, AdUIIR-infected cardiomyocytes were transfected with the ANF, SkA, or MEF2 reporter plasmids and were treated with 250 ng/ml STO-609 for 1 h prior to UII treatment. Pretreatment of cells with STO-609 completely blocked the ability of UII to activate the hypertrophy marker gene reporters, demonstrating the necessity of CaMKK for UII-mediated effects on hypertrophy marker gene expression and MEF2 activity (FIG. 2A).

UII stimulation of mRNA for ANF, BNP, αMHC and SkA is dependent on CaMKK. To demonstrate further that UII promotes hypertrophy through CaMKK, we examined changes in the expression of mRNA encoding hypertrophy marker genes in cardiomyocytes cultured in the absence or presence of UII and STO-609. Untreated cardiomyocytes showed only low level expression of the four definitive hypertrophy marker genes, ANF, BNP, and αMHC, and SkA as measured by RT-PCR (FIG. 2B). In contrast, cells treated with UII for 48 h displayed increased expression of all four hypertrophy marker genes (FIG. 2B). In the presence of STO-609, UII was unable to stimulate hypertrophy marker gene expression over control. When combined, the results from FIG. 2 show that UII stimulates hypertrophic-sensitive marker gene expression through CaMKK.

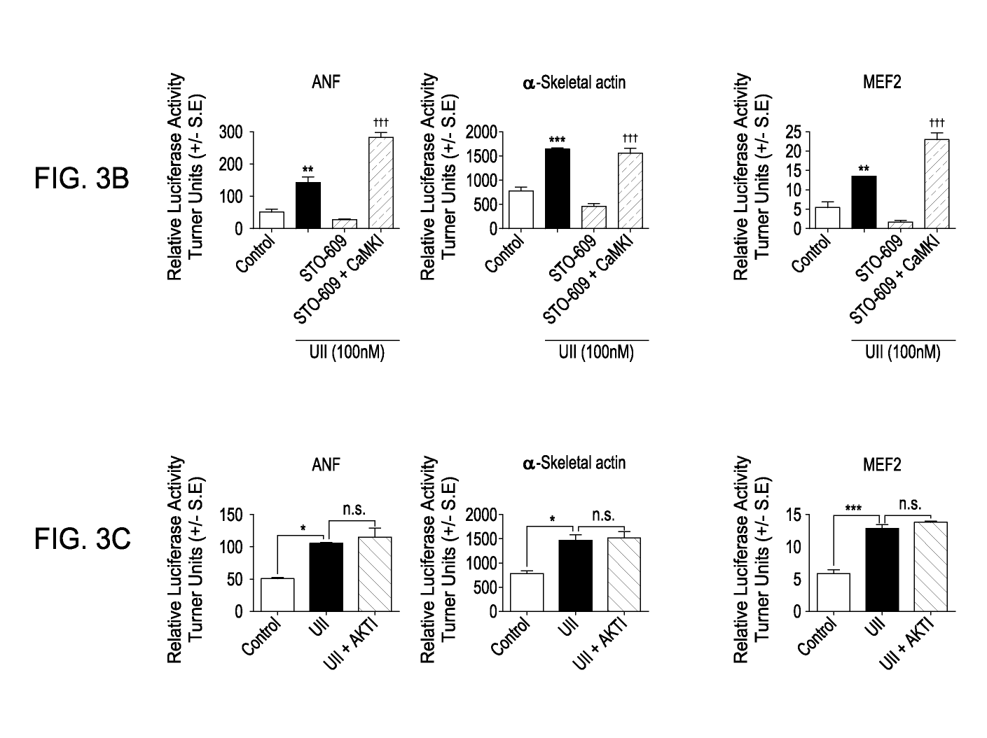

UII stimulation results in the activation of CaMKI. The data shown in FIG. 2 demonstrate that CaMKK is required for UII-induced hypertrophic gene expression in cardiomyocytes. One of the major downstream effectors of CaMKK is CaMKI. In addition to calcium/calmodulin, CaMKI requires phosphorylation by CaMKK to be active. To determine whether UII activates CaMKI, AdUIIR-infected cardiomyocyte cultures were treated with UII for various times up to 60 min, and whole cell lysates were collected and the phosphorylation status of CaMKI was measured by Western blot analysis by using an antibody that recognizes the active form of CaMKI (phospho-Thr178). Two minutes of UII treatment was sufficient to increase the relative amount of active CaMKI compared with untreated cells (FIG. 3A). CaMKI remained phosphorylated and active through 60 min of UII stimulation. Thus, UII activates CaMKI in cardiomyocytes.

To demonstrate that UII activates CaMKI through CaMKK, cardiomyocytes were cultured in the absence or presence of UII and STO-609 for 2 or 5 min. Untreated cardiomyocytes showed no activation of CaMKI while UII stimulation resulted in activation of CaMKI as seen previously. In contrast, UII was unable to stimulate CaMKI in the presence of STO-609 (FIG. 3A). In addition, constitutively-active CaMKI was able to rescue UII-stimulated ANF, SkA and MEF2 reporter activities while CaMKK was inhibited (FIG. 3B). To exclude the possibility of AKT acting downstream of CaMKK, we cultured cardiomyocytes in the absence or presence of UII and an AKT inhibitor. AKT inhibition had no effect on the ability of UII to stimulate ANF, SkA and MEF2 reporters (FIG. 3C). Taken together, these data demonstrate that UII specifically activates CaMKI through CaMKK and stimulation of hypertrophic reporter activity is not dependent on AKT.

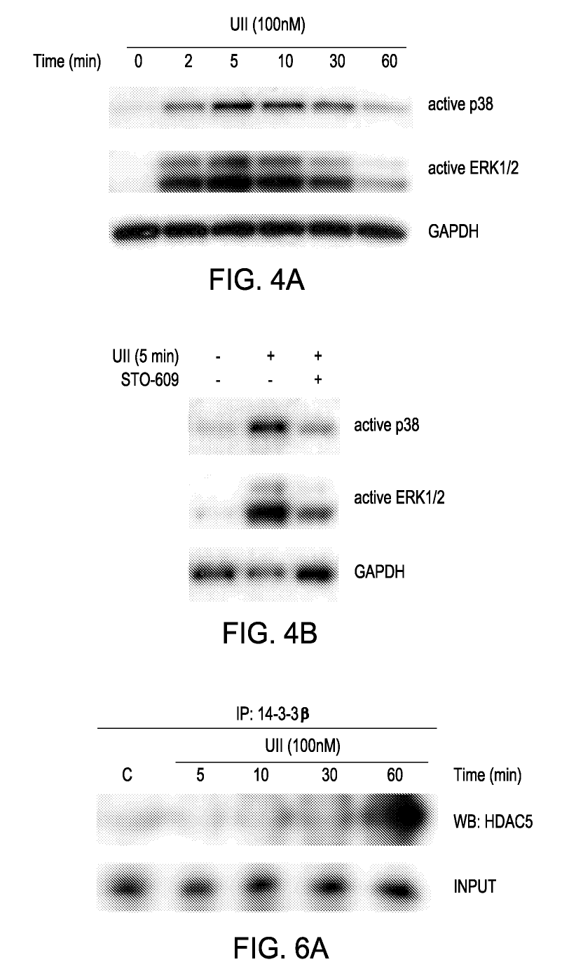

UII-dependent activation of p38 and ERK1/2 requires CaMKK. MAP kinases play an important role in mediating intracellular signaling. They are involved in cellular processes such as hypertrophy (7-10, 53, 54). More interesting, cross talk has been reported between the CaMKs and the MAPKs in neurons (11). There are three distinct families of MAP kinases (ERKs, p38s, and JNKs)(55). UII has been shown previously to activate p38 and ERK1/2 MAPKs in cardiomyocytes (44). Next, whether UII stimulation of the MAP kinases was dependent on CaMKK was determined. AdUIIR-infected cardiomyocyte cultures were treated with UII for various times up to 60 min, and the phosphorylation status of p38 and ERK MAPKs were measured by Western blot analysis using antibodies that detect the active (phosphorylated) forms of these kinases. Phosphorylation of p38 and ERK1/2 increased in a time-dependent manner following UII treatment as described previously (FIG. 4A). To demonstrate that UII activates p38 and ERK1/2 through CaMKK, cardiomyocytes were cultured in the absence or presence of UII and STO-609 for 5 min. Untreated cardiomyocytes showed no activation of p38 or ERK1/2 while UII stimulation resulted in phosphorylation of p38 and ERK1/2 as seen previously. In contrast, UII was unable to stimulate p38 or ERK1/2 in the presence of STO-609 (FIG. 4B). TO our knowledge, this is the first demonstration of CaMK to MAPK crosstalk in cardiomyocytes.

MAP kinase inhibition results in the inability of UII or CaMKI to stimulate ANF, SkA and MEF2 reporter activities. Signaling through Gq results in hypertrophic gene expression in cardiomyocytes and is dependent on MAP kinase activation (56, 57). Since UII is able to induce hypertrophic gene expression through a Gq mechanism, whether inhibition of p38 and ERK1/2 would result in the loss of UII- and CaMKI-dependent ANF, SkA and MEF2 reporter activity was determined. One set of cardiomyocytes were AdUIIR-infected and the other co-transfected with CaMKI and either ANF, SkA, or MEF2 reporter plasmids and were treated with SB203580 or U0126 as indicated for 1 h prior to stimulation. Pretreatment of cells with SB203580 or U0126 completely blocked the ability of UII or CaMKI to activate the hypertrophy reporters, demonstrating the necessity of p38 and ERK1/2 (FIGS. 4C and 4D). When combined, the results from FIG. 4 shows that the CaMK and MAPK pathways crosstalk to regulate hypertrophy-sensitive gene expression through p38 and ERK1/2. These data are the first to demonstrate this important mechanism in cardiomyocytes.

Dominant negative p38 inhibits UII and CaMKI stimulation of MEF2. p38 is a known activator of MEF2 (18). To further determine whether p38 MAP kinase is required for UII- or CaMKI-mediated activation of MEF2, we examined whether a dominant negative p38 MAP kinase mutant could block the ability of either UII or CaMKI to activate the MEF2 reporter. For stimulation, cardiomyocytes were either infected with AdUIIR and treated with UII, or transfected with CaMKI. All cultures were also transfected with the MEF2 reporter and dominant negative p38, and the activity of the MEF2 enhancer was measured. UII and CaMKI increased the activity of each enhancer, as expected (FIGS. 5A and 5B). Co-expression of the dominant negative p38 MAP kinase abolished the ability of UII or CaMKI to increase MEF2 activity (FIGS. 5A and 5B). These data further demonstrate the necessity of p38 MAP kinase for UII-mediated activation of MEF2 and more important, demonstrate that CaMKI requires p38 for MEF2 activation.

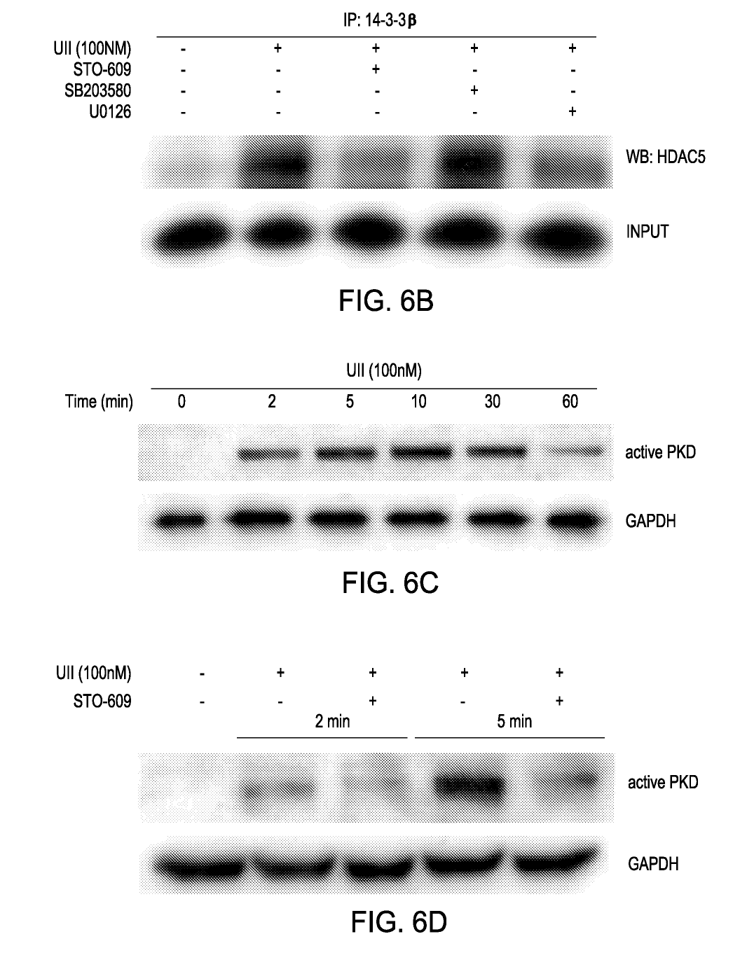

UII stimulation induces association of HDAC5 with 14-3-3β in a CaMKK dependent manner. The activity of MEF2 is controlled, in part, by its association with class II HDACs in the nucleus. Class II HDACs repress the activity of MEF2-sensitive promoters by local deacetylation of nucleosomal histones causing the condensation of chromatin. The repressive influence of HDACs on MEF2 can be relieved by CaMK-dependent phosphorylation of HDACs, which results in their dissociation from MEF2. The dissociation of HDACs from MEF2 is accompanied by 14-3-3-mediated nuclear export of the HDACs. We have shown that UII stimulates the activity of MEF2. Next, to determine whether UII stimulates HDAC5 association with 14-3-3β, AdUIIR-infected cardiomyocytes were treated with UII for various times up to 60 min, and 14-3-3β was pulled down from the whole cell lysate by using an antibody specific for 14-3-3β and protein G agarose. HDAC5 was detected using a specific antibody. Sixty minutes of UII treatment was sufficient to increase the relative amount of 14-3-3β-bound HDAC5 compared with untreated cells (FIG. 6A). The input 14-3-3β did not change over UII stimulation time. These data clearly demonstrate the ability of UII to stimulate the association of HDAC5 with 14-3-3β.

To determine the effects of CaMKK and MAP kinase inhibition on the UII-induced association of HDAC5 with 14-3-3β the following study was conducted. AdUIIR-infected cardiomyocytes were pretreated with STO-609, SB203580 or U0126 1 h prior to the addition of UII and 14-3-3β was immunoprecipitated as before and HDAC5 detected. Inhibition of either CaMKK or ERK1/2 resulted in a loss of UII-induced association of HDAC5 with 14-3-3β while inhibition of p38 had no effect (FIG. 6B). Importantly, these data demonstrate that UII-stimulated HDAC5/14-3-3β association is dependent on CaMKK. These data provide a mechanism whereby UII stimulation of CaMKK is sufficient to activate MEF2 (likely via phosphorylation by p38) and relieve HDAC5 association with MEF2—two events that are required for activation of MEF2.

UII activates PKD in a CaMKK dependent manner. A parallel pathway involving PKD phosphorylatea HDACs resulting in their 14-3-3-dependent translocation to the nucleus (19). All of our previous work with the HDACs employed active CaMKs (I and IV) or phenylephrine to induce HDAC nucleocytoplasmic shuttling 126. The present inventors have shown that UII is able to induce the association of HDAC5 with 14-3-3β in a CaMKK dependent manner. CaMKI is a cytoplasmic kinase and is activated by UII stimulation. Next, it was determined whether UII stimulation could activate PKD which would account for the UII-induced HDAC5 translocation to the nucleus possibly working in conjunction with CaMKI. To determine whether UII activates PKD, AdUIIR-infected cardiomyocyte cultures were treated with UII for various times up to 60 min, and whole cell lysates were collected and the phosphorylation status of PKD was measured by Western blot analysis by using an antibody that recognizes the active form of PKD (phospho-Ser744/748). UII stimulation resulted in the phosphorylation of PKD by two minutes with maximum phosphorylation observed at 10 minutes (FIG. 6C). By 60 minutes, phosphorylation of PKD returned to near basal.

To determine whether UII activates PKD through CaMKK, cardiomyocytes were cultured in the absence or presence of UII and STO-609 for 2 or 5 min. Untreated cardiomyocytes showed no activation of PKD while UII stimulation resulted in phosphorylation of PKD as seen previously. In contrast, UII was unable to stimulate PKD in the presence of STO-609 (FIG. 6D). These data are the first to show that UII is capable of stimulating the activation of PKD. More important, this is the first demonstration that CaMK is required for the activation of PKD and may account for the CaMKK-dependent nucleocytoplasmic shuttling of HDAC5 and may account for the CaMKK-dependent nucleocytoplasmic shuttling of HDAC5.

UII is capable of inducing the hypertrophic phenotype in cultured cardiomyocytes. It has been shown that UII stimulation of cardiomyocytes results in increased cell size, increased protein to DNA ratio and sarcomeric reorganization (58). Presently, the signaling mechanisms that couple UII to hypertrophy are not completely known.

The expression of UIIR is undetectable to slight in healthy myocardium. It is only in states of myocardial disease or dysfunction that UIIR expression becomes markedly up-regulated (38, 41-43). The etiology of heart disease and failure nearly always includes cardiac hypertrophy. UII is known to induce hypertrophy in cell culture, but only when the UIIR is sufficiently expressed. Sustained mechanical stress to the myocardium often leads to cardiac hypertrophy. There is evidence which suggests that mechanical stress itself increases the availability of humoral factors known to induce myocyte hypertrophy; for instance, AngII (25, 26, 28). It is possible that at some point during the etiology of heart failure, available hypertrophic agonists stimulate the expression of UIIR over the necessary threshold for UII to elicit its biological activity thereby contributing to a downward spiral toward dilated cardiomyopathy and failure.

Next, the inventors determined whether known inducers of cardiac hypertrophy are capable of up-regulating UIIR in cardiomyocytes. Specific gene primers were designed for rat UIIR and replicated the array study. Through the use of RT-PCR, it was demonstrated that stimulation of cardiomyocytes with PE and AngII results in an increase of UIIR mRNA (FIG. 1A). Corresponding with the increase of UIIR mRNA, it is also shown herein that that PE and AngII stimulation of cardiomyocytes results in the increase of UIIR protein (FIG. 1B).

It was found that signaling through Gq-coupled receptors increases the expression of other Gq-coupled receptors; for example, Angiotensin II stimulates the up-regulation of Endothelin receptor B (ETBR) in cardiomyocytes (30). It has been observed that moderate degrees of Gq signaling stimulate adaptive hypertrophy (31-33), whereas high degrees of Gq signaling result in maladaptive cardiomyocyte apoptosis (34-37). These results demonstrate that hypertrophy-stimulating humoral factors such as AngII result in the up-regulation of another Gq-coupled receptor, UIIR.

Signaling through the UIIR is known to activate PLC producing IP3 and DAG. IP3 activates the sarcoplasmic reticulum IP3 receptor that releases Ca²⁺ to the cytoplasm. However, Onan et al. showed that inhibition of PKC does not result in the inhibition of UII-mediated hypertrophy induction (44). The complete signaling pathway through UIIR has not been fully delineated; however, others have shown that UII stimulation activates members of the MAP kinase pathway. The results shown herein demonstrate that UII stimulation of MEF2 and ANF is dependent on CaMKK activity. CaMKI is immediately downstream of and requires CaMKK to be active. CaMKI has been shown to activate ERK 1/2 (11) and UII stimulation activates both ERK1/2 and p3878. Therefore, ERK 1/2 and p38 are activated through UIIR and are dependent on CaMKI activation by CaMKK.

Further studies were conducted with cardiomyocytes that were stimulated with varying doses of UII and ANF reporter activity was measured. Under the culture conditions, cardiomyocytes were incompetent to respond to UII (data not shown). These results are consistent with the fact that the expression of UIIR in normal rat cardiomyocytes is slight to nonexistent. In addition, these results show that UII is incapable of stimulating cardiomyocytes through a non-receptor mediated mechanism. In order to study the hypertrophic effects of UII in a cell culture model system, adenoviral delivery of UIIR was used.

Data from the present study clearly demonstrate that UII stimulation of cardiomyocytes expressing UIIR results in the induction of hypertrophy marker genes. This was first shown by promoter reporter assays in which cardiomyocytes were transfected with either ANF, SkA or MEF2 reporters (data not shown). Since UII elicits the mobilization of intracellular calcium through classical Gq coupling mechanisms, UII stimulation of hypertrophy gene induction could include the involvement of CaMKK. In order to demonstrate this, promoter reporter assays were used to measure the activities of the ANF, SkA and MEF2 plasmid reporters. In the presence of STO-609, a potent and selective inhibitor of CaMKK, UII was incapable of stimulating any of the reporters (FIG. 2A). Importantly, the selectivity of STO-609 for CaMKK has an IC₅₀ value is 120 ng/mL for CaMKKa and 40 ng/mL for CaMKKb. Other kinases are inhibited by STO-609 (CaM-KII, MLCK (IC₅₀ ˜10 mg/mL), CaM-KI, CaM-KIV, PKA, PKC, and p42 MAP kinase (IC₅₀>10 μg/mL)), but only well above the dose used in the current study (250 ng/mL). In addition, the ability of UII to increase the message of several hypertrophy-sensitive genes in the cardiomyocyte we determined. It was found that UII was able to increase the expression of all hypertrophy marker genes studied. However, in the presence of STO-609, UII was unable to stimulate the expression of ANF, BNP, αMHC or SkA (FIG. 2B). These data, for the first time, implicate CaMKK as a major component in UII-induced hypertrophic gene induction.

Through the use of western blots, it was determined that UII stimulation of cardiomyocytes results in a time-dependent activation of CaMKI (FIG. 3A (left panel)). These results indicated for the first time that CaMKI is a major downstream effector of CaMKK. Indeed, in the presence of STO-609, the UII-dependent activation of CaMKI was inhibited (FIG. 3A (right panel)). The activation of CaMKI by UII provides a critical insight into the mechanism by which UII is able to elicit a hypertrophic response in cardiomyocytes. The present inventors have previously demonstrated that constitutively active CaMKI transfection of cardiomyocytes results in a robust activation of ANF and SkA reporters, MEF2 activity as well as the 14-3-3 dependent translocation of class II HDACs to the cytoplasm. The constitutively active mutant of CaMKI is active independent of Ca²⁺/CaM and CaMKK. It is shown herein that constitutively active CaMKI completely rescues UII stimulation of ANF, SkA and MEF2 when CaMKK is inhibited (FIG. 3B). These results demonstrate that under UII stimulation, CaMKI is active and is a major component of the downstream signaling from the UII receptor. The ability of CaMKI to rescues UII stimulation under conditions where CaMKK was inhibited further implicates the CaM kinase pathway as necessary for UII-dependent hypertrophy gene induction. If an additional signaling component downstream of CaMKK was necessary for full UII stimulation, while not expected that CaMKI alone could fully recapitulate UII stimulation of ANF, SkA and MEF2. To date, only three kinases are known downstream targets of CaMKK: CaMKI, CaMKIV and AKT. Of these, only CaMKI and AKT are expressed in the heart. These results demonstrate the role of CaMKI, however AKT may also be involved. Next, it was determined whether inhibition of AKT could affect UII stimulation of hypertrophy-sensitive promoters. It was found that no reduction of UII stimulation of ANF, SkA and MEF2 reporter activities when AKT was inhibited (FIG. 3C). These AKT data are in agreement with others who have shown that inhibition of PI3K had no effect on UII stimulation of hypertrophy (44).

It is known that UII stimulation of cardiomyocytes results in a time-dependent activation of p38 and ERK1/2 MAP kinases (44). In fact, several studies demonstrate the overall importance of p38 (59-62) and ERK1/2 (9, 57, 60, 63) in the progression of cardiomyocyte hypertrophy both in vivo and in vitro. Onan, et al. demonstrated that UII stimulation of p38 and ERK1/2 activities might be dependent on the ability of the UIIR to transactivate the EGFR. Additionally, UII induced hypertrophy was completely prevented only if the EGFR and ERK1/2 were inhibited. These data led the authors to suggest that there may be an EGFR independent pathway leading to ERK1/2 activation and myocyte hypertrophy. In other cell types, Ca²⁺ is capable of activating both MAPKs (11). More important, Ca²⁺ activation of MAPKs is dependent on the CaMK cascade. Most of the work showing this was done using neuronal cells lines—excitable cells like cardiomyocytes. In line with this, we hypothesized that UII stimulation of cardiomyocytes resulting in the activation of p38 and ERK1/2 is dependent on CaMKK. The inventors have already shown that UII stimulation of cardiomyocytes resulted in a time-dependent activation of CaMKI. Using antibodies that detect only the active forms of p38 and ERK1/2, we determined by western blot analysis that both p38 and ERK1/2 were indeed activated in a time-dependent manner by UII stimulation of this cardiomyocyte cell model (FIG. 4A). These data confirmed the findings by Onan, et al.

It was found herein that when CaMKK was inhibited, UII stimulation of active p38 and ERK1/2 was almost completely repressed (FIG. 4B). The portion of activated p38 and ERK1/2 that remained after CaMKK inhibition may be due to EGFR transactivation by UII as seen by Onan, et al. However, the present inventors recognized that CaMKK inhibition completely blocked UII activation of ANF, SkA and MEF2 in earlier studies. It is probable that MAP kinase activation must meet a threshold of activity prior to stimulating hypertrophy marker genes and the UII-induced EGFR component is not capable of reaching this threshold. An interesting note must be made regarding the EGF receptor. It has been shown that CaM is capable of binding EGFR and thus inhibits PKC-dependent activation (64). Under the conditions of these studies, the dependence of CaMKK—which requires CaM—as well as the activation of CaMKI was demonstrated. Onan, et al., found that UII stimulation of hypertrophy did not require PKC activity. Importantly, it was show that p38 and ERK1/2 activation by UII did not depend on PKC. A mechanism that accounts for the fact that UII activation of MAP kinases and hypertrophy does not require PKC. Namely, it was shown herein that UII stimulates hypertrophy marker gene expression and MAP kinase activation through CaMKK and consequently, through CaMKI.

In the present studies it was found that inhibition of p38 or ERK1/2 resulted in a complete loss of the ability of UII to stimulate ANF, SkA and MEF2 reporter activities (FIG. 4C). More important, it is demonstrated that CaMKI stimulation of ANF, SkA and MEF2 was also prevented when p38 and ERK1/2 were inhibited (FIG. 4D). Taken together, these data demonstrate a heretofore unknown novel crosstalk system between the CaM kinase cascade and MAP kinases not previously described in cardiomyocytes. This crosstalk mechanism not only helps to explain UII-dependent stimulation of cardiomyocyte hypertrophy, but is relevant for other Gq-coupled receptors such as for AngII. Indeed, in additional studies it was found that the AngII- or ET-1-dependent activation of ERK1/2 also required CaMKK (FIG. 7A).

As p38 MAPKs require dual phosphorylation of both a Thr and a Tyr residue to be active, it is improbable that CaMKI directly activates p38. It is possible that CaMKK or CaMKI acts somewhere upstream of p38 to cause its activation and should therefore be explored.

MEF2 is a transcription factor that is a major component of the signaling involved with cardiomyocyte hypertrophy. MEF2 is critical for the development of the heart. In the adult heart, MEF2 is held inactive by class II HDACs. Under conditions that result in cardiomyocyte hypertrophy, HDACs are phosphorylated and transported to the cytoplasm via 14-3-3 proteins, thereby relieving repression of MEF2. We have previously demonstrated that CaMKI is capable of stimulating the phosphorylation and nucleocytoplasmic shuttling of HDAC5 resulting in MEF2 transcriptional activity (65). In addition to the removal of HDACs, the activation of MEF2 is dependent on phosphorylation by p38 (18).

Using a dominant negative p38, it was demonstrated that the UII-dependent activation of MEF2 required functional p38 (FIG. 5A). In addition, it is shown that CaMKI-dependent activation of MEF2 also required p38 (FIG. 5B). These data corroborate our earlier findings using a pharmacological inhibitor of p38 whereby either UII or CaMKI stimulation of MEF2 activity was abolished by p38 inhibition. More important, it was found that the stimulation of cardiomyocytes with UII resulted in the time-dependent association of HDAC5 with 14-3-3β (FIG. 6A). In addition, inhibition of CaMKK with STO-609 resulted in a decreased association of HDAC5 with 14-3-3β in cardiomyocytes stimulated with UII (FIG. 6B). Inhibition of p38 had no effect on HDAC5 association with 14-3-3β under UII stimulation. This was to be expected as p38 activates MEF2 directly through phosphorylation whereas p38 phosphorylation of HDACs has never been described. Not wanting to be bound by theory it show two independent mechanisms of MEF2 activation by UII: i) relief of HDAC repression and ii) activation of MEF2 by p38. When ERK1/2 was inhibited with U0126, there was a reduced UII-dependent association with 14-3-3β.

Not wanting to be bound by theory, these results suggest that ERK1/2 may be able to regulate HDAC5. In fact, the association of ERK1/2 with HDAC4 has been reported; however, it appears that ERK1/2 kinase activity induces nuclear localization of HDAC4 (66). U0126 also inhibits ERK5. ERK5 was shown to interact with MEF2 (67). Whether ERK5 is capable of phosphorylating HDACs remains to be seen and should be explored.

In cardiomyocytes, two parallel pathways have been proposed which result in the export of class II HDACs from the nucleus. The first pathway is through CaMKI as we have shown, and the second is through PKD (19). PKD was shown to directly phosphorylate HDAC5 resulting in its nuclear export. Since UII was able to induce the association of HDAC5 with 14-3-3β, we could not rule out the possibility that PKD was involved. Using an antibody specific for active PKD phosphorylated at Ser744 and Ser748, it was demonstrated that UII stimulation of cardiomyocytes resulted in the time-dependent activation of PKD (FIG. 6C). More interesting, it is also demonstrated herein that CaMKK inhibition blocked the UII-dependent phosphorylation of PKD (FIG. 6D). Again, not wishing to be bound by theory, PKD may be downstream of CaMKK or CaMKI. To date, no kinase activity has been proposed downstream of CaMKI that results in the export of HDAC from the nucleus. CaMKI is localized to the cytoplasm and has not been reported in the nucleus. Again, not wishing to be bound by theory, it is possible that the CaM kinase cascade activates PKD resulting in HDAC nuclear export. Indeed, the data suggests that activation of PKD by AngII and Et-1 requires CaMKK (FIG. 7B).

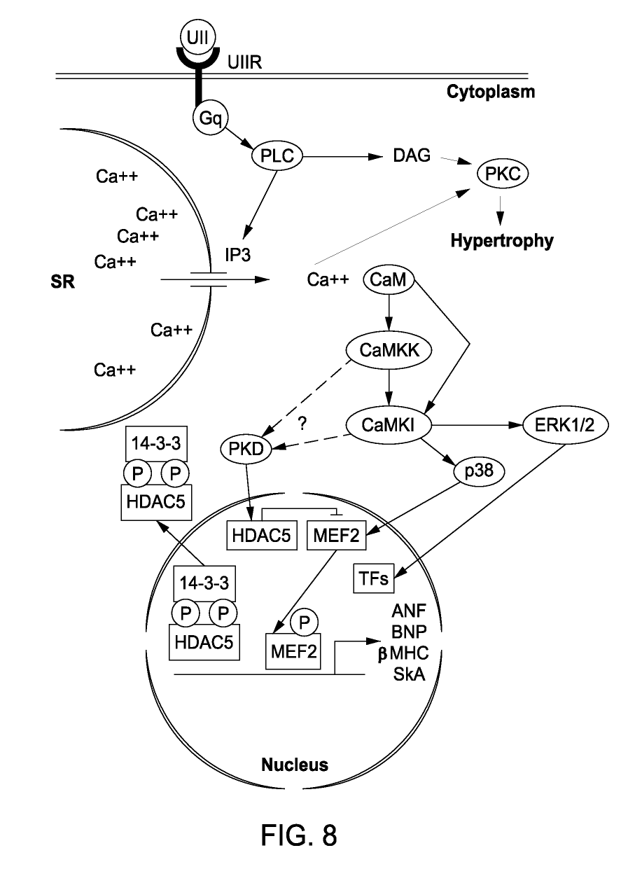

The results presented here clearly demonstrate for the first time an important role for CaMKK in UII-mediated cardiomyocyte hypertrophy. UII was able to stimulate the promoter activity of ANF and SkA and the transcriptional activity of MEF2 in a CaMKK-dependent manner. UII stimulation of ANF, BNP, αMHC and SkA gene expression was dependent on CaMKK. UII stimulation caused the CaMKK-dependent activation of CaMKI. Constitutively-active CaMKI completely rescued UII stimulation of ANF and SkA promoter activities as well as MEF2 activity with CaMKK pharmacologically inhibited. The inhibition of AKT had no effect on the ability of UII to stimulate hypertrophy-sensitive promoters or MEF2 activity. The inventors also demonstrate that the UII-induced activation of p38 and ERK1/2 MAP kinases was dependent on CaMKK suggesting a novel cross-talk mechanism not previously described in cardiomyocytes. Both UII- and CaMKI-mediated induction of ANF, SkA and MEF2 reporter activities was dependent on p38 and ERK1/2. These data allow the inventors to construct a more complete pathway whereby UII stimulation results in hypertrophic gene induction. Moreover, this new pathway involves a crosstalk mechanism between the CaMKs and the MAPKs not previously reported in cardiomyocytes (FIG. 8).

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Ray, A., Aumont, M. C., Aussedat, J., Bercovici, J., Rossi, A., and Swynghedauw, B. (1987) Cardiovasc Res 21(8), 587-592
2. Schreiber, S. S., Evans, C. D., Oratz, M., and Rothschild, M. A. (1981) Circ Res 48(5), 601-611
3. Mercadier, J. J., Samuel, J. L., Michel, J. B., Zongazo, M. A., de la Bastie, D., Lompre, A. M., Wisnewsky, C., Rappaport, L., Levy, B., and Schwartz, K. (1989) Am J Physiol 257(3 Pt 2), H979-987
4. Saito, Y., Nakao, K., Arai, H., Nishimura, K., Okumura, K., Obata, K., Takemura, G., Fujiwara, H., Sugawara, A., Yamada, T., and et al. (1989) J Clin Invest 83(1), 298-305
5. Schiaffino, S., Samuel, J. L., Sassoon, D., Lompre, A. M., Garner, I., Marotte, F., Buckingham, M., Rappaport, L., and Schwartz, K. (1989) Circ Res 64(5), 937-948
6. Schwartz, K., de la Bastie, D., Bouveret, P., Oliviero, P., Alonso, S., and Buckingham, M. (1986) Circ Res 59(5), 551-555
7. Bogoyevitch, M. A., Glennon, P. E., Andersson, M. B., Clerk, A., Lazou, A., Marshall, C. J., Parker, P. J., and Sugden, P. H. (1994) J Biol Chem 269(2), 1110-1119
8. Clerk, A., Bogoyevitch, M. A., Anderson, M. B., and Sugden, P. H. (1994) J Biol Chem 269(52), 32848-32857
9. Post, G. R., Goldstein, D., Thuerauf, D. J., Glembotski, C. C., and Brown, J. H. (1996) J Biol Chem 271(14), 8452-8457
10. Yamazaki, T., To be, K., Hoh, E., Maemura, K., Kaida, T., Komuro, I., Tamemoto, H., Kadowaki, T., Nagai, R., and Yazaki, Y. (1993) J Biol Chem 268(16), 12069-12076
11. Enslen, H., Tokumitsu, H., Stork, P. J., Davis, R. J., and Soderling, T. R. (1996) Proc Natl Acad Sci USA 93(20), 10803-10808
12. Naya, F. J., and Olson, E. (1999) Curr Opin Cell Biol 11(6), 683-688
13. Passier, R., Zeng, H., Frey, N., Naya, F. J., Nicol, R. L., McKinsey, T. A., Overbeek, P., Richardson, J. A., Grant, S. R., and Olson, E. N. (2000) J Clin Invest 105(10), 1395-1406
14. Lernercier, C., Verdel, A., Galloo, B., Curtet, S., Brocard, M. P., and Khochbin, S. (2000) J Biol Chem 275(20), 15594-15599
15. Lu, J., McKinsey, T. A., Nicol, R. L., and Olson, E. N. (2000) Proc Natl Acad Sci USA 97(8), 4070-4075
16. Miska, E. A., Karlsson, C., Langley, E., Nielsen, S. J., Pines, J., and Kouzarides, T. (1999) Embo J 18(18), 5099-5107
17. Youn, H. D., Grozinger, C. M., and Liu, J. O. (2000) J Biol Chem 275(29), 22563-22567
18. Han, J., Jiang, Y., Li, Z., Kravchenko, V. V., and Ulevitch, R. J. (1997) Nature 386(6622), 296-299
19. Vega, R. B., Harrison, B. C., Meadows, E., Roberts, C. R., Papst, P. J., Olson, E. N., and McKinsey, T. A. (2004) Mol Cell Biol 24(19), 8374-8385
20. Davis, F. J., Gupta, M., Camoretti-Mercado, B., Schwartz, R. J., and Gupta, M. P. (2003) J Biol Chem 278(22), 20047-20058
21. Kennedy, R. A., Kemp, T. J., Sugden, P. H., and Clerk, A. (2006) J Mol Cell Cardiol 41(2), 236-247
22. McDonough, P. M., Stella, S. L., and Glembotski, C. C. (1994) J Biol Chem 269(13), 9466-9472
23. Pellieux, C., Sauthier, T., Aubert, J. F., Brunner, H. R., and Pedrazzini, T. (2000) J Hypertens 18(9), 1307-1317
24. Arai, M., Yoguchi, A., Iso, T., Takahashi, T., Imai, S., Murata, K., and Suzuki, T. (1995) Am J Physiol 268(5 Pt 2), H2084-2091
25. Miyata, S., Haneda, T., Osaki, J., and Kikuchi, K. (1996) Eur J Pharmacol 307(1), 81-88
26. Sadoshima, J., Xu, Y., Slayter, H. S., and Izumo, S. (1993) Cell 75(5), 977-984
27. Semeri, G. G., Modesti, P. A., Boddi, M., Cecioni, I., Paniccia, R., Coppo, M., Galanti, G., Simonetti, I., Vanni, S., Papa, L., Bandinelli, B., Migliorini, A., Modesti, A., Maccherini, M., Sani, G., and Toscano, M. (1999) Circ Res 85(1), 57-67
28. Tamura, K., Umemura, S., Nyui, N., Hibi, K., Ishigami, T., Kihara, M., Toya, Y., and Ishii, M. (1998) Am J Physiol 275(1 Pt 2), R1-9
29. Yamazaki, T., Komuro, I., Kudoh, S., Zou, Y., Shiojima, I., Hiroi, Y., Mizuno, T., Maemura, K., Kurihara, H., Aikawa, R., Takano, H., and Yazaki, Y. (1996) J Biol Chem 271(6), 3221-3228
30. Hirata, Y., Kanno, K., Eguchi, S., and Kano, H. (1994) Blood Press Suppl 5, 84-88
31. D'Angelo, D. D., Sakata, Y., Lorenz, J. N., Boivin, G. P., Walsh, R. A., Liggett, S. B., and Dom, G. W., 2nd. (1997) Proc Natl Acad Sci USA 94(15), 8121-8126
32. LaMorte, V. J., Thorburn, J., Absher, D., Spiegel, A., Brown, J. H., Chien, K. R., Feramisco, J. R., and Knowlton, K. U. (1994) J Biol Chem 269(18), 13490-13496
33. Milano, C. A., Dolber, P. C., Rockman, H. A., Bond, R. A., Venable, M. E., Allen, L. F., and Lefkowitz, R. J. (1994) Proc Natl Acad Sci USA 91(21), 10109-10113
34. Adams, J. W., Pagel, A. L., Means, C. K., Oksenberg, D., Armstrong, R. C., and Brown, J. H. (2000) Circ Res 87(12), 1180-1187
35. Adams, J. W., Sakata, Y., Davis, M. G., Sah, V. P., Wang, Y., Liggett, S. B., Chien, K. R., Brown, J. H., and Dorn, G. W., 2nd. (1998) Proc Natl Acad Sci USA 95(17), 10140-10145
36. Althoefer, H., Eversole-Cire, P., and Simon, M. I. (1997) J Biol Chem 272(39), 24380-24386
37. Sakata, Y., Hoit, B. D., Liggett, S. B., Walsh, R. A., and Dorn, G. W., 2nd. (1998) Circulation 97(15), 1488-1495
38. Zhang, Y., Li, J., Cao, J., Chen, J., Yang, J., Zhang, Z., Du, J., and Tang, C. (2002) Heart Vessels 16(2), 64-68
39. Matsushita, M., Shichiri, M., Imai, T., Twashina, M., Tanaka, H., Takasu, N., and Hirata, Y. (2001) J Hypertens 19(12), 2185-2190
40. Ames, R. S., Sarau, H. M., Chambers, J. K., Willette, R. N., Aiyar, N. V., Romanic, A. M., Louden, C. S., Foley, J. J., Sauermelch, C. F., Coatney, R. W., Ao, Z., Disa, J., Holmes, S. D., Stadel, J. M., Martin, J. D., Liu, W. S., Glover, G. I., Wilson, S., McNulty, D. E., Ellis, C. E., Elshourbagy, N. A., Shabon, U., Trill, J. J., Hay, D. W., Ohlstein, E. H., Bergsma, D. J., and Douglas, S. A. (1999) Nature 401(6750), 282-286
41. Douglas, S. A., Tayara, L., Ohlstein, E. H., Halawa, N., and Giaid, A. (2002) Lancet 359(9322), 1990-1997
42. Tzanidis, A., Hannan, R. D., Thomas, W. G., Onan, D., Autelitano, D. J., See, F., Kelly, D. J., Gilbert, R. E., and Krum, H. (2003) Circ Res 93(3), 246-253
43. Zhou, P., Wu, S. Y., Yu, C. F., Wang, H., Tang, C. S., Lin, L., and Yuan, W. J. (2003) Sheng Li Xue Bao 55(4), 442-448
44. Onan, D., Pipolo, L., Yang, E., Hannan, R. D., and Thomas, W. G. (2004) Mol Endocrinol 18(9), 2344-2354
45. Toraason, M., Luken, M. E., Breitenstein, M., Krueger, J. A., and Biagini, R. E. (1989) Toxicology 56(1), 107-117
46. McBride, K., Robitaille, L., Tremblay, S., Argentin, S., and Nemer, M. (1993) Mol Cell Biol 13(1), 600-612
47. MacLellan, W. R., Lee, T. C., Schwartz, R. J., and Schneider, M. D. (1994) J Biol Chem 269(24), 16754-16760
48. Wu, H., Rothermel, B., Kanatous, S., Rosenberg, P., Naya, F. J., Shelton, J. M., Hutcheson, K. A., DiMaio, J. M., Olson, E. N., Bassel-Duby, R., and Williams, R. S. (2001) Embo J 20(22), 6414-6423
49. McKinsey, T. A., Zhang, C. L., and Olson, E. N. (2000) Proc Natl Acad Sci USA 97(26), 14400-14405
50. Han, J., Lee, J. D., Jiang, Y., Li, Z., Feng, L., and Ulevitch, R. J. (1996) J Biol Chem 271(6), 2886-2891
51. Li, Z., Jiang, Y., Ulevitch, R. J., and Han, J. (1996) Biochem Biophys Res Commun 228(2), 334-340
52. Raingeaud, J., Gupta, S., Rogers, J. S., Dickens, M., Han, J., Ulevitch, R. J., and Davis, R. J. (1995) J Biol Chem 270(13), 7420-7426
53. Rapacciuolo, A., Esposito, G., Caron, K., Mao, L., Thomas, S. A., and Rockman, H., A. (2001) J Am Coll Cardiol 38(3), 876-882
54. Takeishi, Y., Huang, Q., Abe, J., Glassman, M., Che, W., Lee, J. D., Kawakatsu, H., Lawrence, E. G., Hoit, B. D., Berk, B. C., and Walsh, R. A. (2001) J Mol Cell Cardiol 33(9), 1637-1648
55. Sugden, P. H., and Clerk, A. (1998) J Mol Med 76(11), 725-746
56. Ueyama, T., Kawashima, S., Sakoda, T., Rikitake, Y., Ishida, T., Kawai, M., Yamashita, T., Ishido, S., Hotta, H., and Yokoyama, M. (2000) J Mol Cell Cardiol 32(6), 947-960
57. Yue, T. L., Gu, J. L., Wang, C., Reith, A. D., Lee, J. C., Mirabile, R. C., Kreutz, R., Wang, Y., Maleeff, B., Parsons, A. A., and Ohlstein, E. H. (2000) J Biol Chem 275(48), 37895-37901
58. Zuo, H. H., Hong, J. M., Guo, H. S., Wei, J. R., He, Y. H., and Li, Z. L. (2004) Di Yi Jun Yi Da Xue Xue Bao 24(6), 642-645
59. Bogoyevitch, M. A. (2000) Cardiovasc Res 45(4), 826-842
60. Ng, D. C., Long, C. S., and Bogoyevitch, M. A. (2001) J Biol Chem 276(31), 29490-29498
61. Sugden, P. H., and Clerk, A. (1998) Circ Res 83(4), 345-352
62. Zechner, D., Thuerauf, D. J., Hanford, D. S., McDonough, P. M., and Glembotski, C. C. (1997) J Cell Biol 139(1), 115-127
63. Esposito, G., Prasad, S. V., Rapacciuolo, A., Mao, L., Koch, W. J., and Rockman, H. A. (2001) Circulation 103(10), 1453-1458
64. Aifa, S., Frikha, F., Miled, N., Johansen, K., Lundstrom, I., and Svensson, S. P. (2006) Biochem Biophys Res Commun 347(2), 381-387
65. Ellis, J. J., Valencia, T. G., Zeng, H., Roberts, L. D., Deaton, R. A., and Grant, S. R. (2003) Mol Cell Biochem 242(1-2), 153-161
66. Zhou, X., Richon, V. M., Wang, A. H., Yang, X. J., Rifkind, R. A., and Marks, P. A. (2000) Proc Natl Acad Sci USA 97(26), 14329-14333
67. Yang, C. C., Ornatsky, O. I., McDermott, J. C., Cruz, T. F., and Prody, C. A. (1998) Nucleic Acids Res 26(20), 4771-4777.

What is claimed is:

1. A composition for treating a cardiac hypertrophy comprising:
an effective amount of a Calmodulin kinase kinase inhibitor sufficient to treat a patient suspected of having cardiac hypertrophy.

2. The composition of claim 1, further comprising a carrier, a diluent, a buffer, a second active agent, a dye, a salt and combinations thereof.

3. The composition of claim 1, wherein the Calmodulin kinase kinase inhibitor comprises a cell-permeable naphthoyl fused benzimidazole compound.

4. The composition of claim 1, wherein the Calmodulin kinase kinase inhibitor is a Calmodulin kinase kinase-specific inhibitor comprises STO-609, derivatives and salts thereof.

5. The composition of claim 1, wherein the Calmodulin kinase kinase inhibitor is provided profilactically to a patient having one or more predisposing factors for cardiac hypertrophy, age-onset cardiomyopathy or angina.

6. The composition of claim 1, wherein the Calmodulin kinase kinase comprises a CaM-KKα, a CaM-KKβ and combinations thereof.

7. The composition of claim 1, wherein the Calmodulin kinase kinase inhibitor comprises 7-H-Benz[de]benzimidazo[2,1-a]isoquinoline-7-one-3-carboxylic acid, acetate.

8. The composition of claim 1, wherein the Calmodulin kinase kinase inhibitor is provided in a dose of at least 120 ng/ml.

9. The composition of claim 1, wherein the Calmodulin kinase kinase inhibitor decreases the expression of Calmodulin kinase kinase selected from an aptamer, an siRNA, a cognate target antagonist or a peptide.

10. The composition of claim 1, wherein the Calmodulin kinase kinase inhibitor comprises a nitric oxide synthase inhibitor.

11. The composition of claim 1, wherein the Calmodulin kinase kinase inhibitor comprises a nitric oxide synthase inhibitor selected from (N-nitro-1-arginine [NNLA], or 7-nitroindazole sodium [7-NINA]).

12. The composition of claim 1, wherein the Calmodulin kinase kinase inhibitor has the formula: 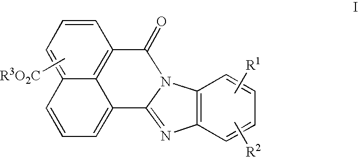
wherein R¹, R²═H, halogen, alkyl, haloalkyl; R³═H, alkyl, substituted alkyl) and their pharmaceutically acceptable salts.

13. The composition of claim 1, wherein the Calmodulin kinase kinase inhibitor is adapted for a dosage of between 0.001 to 500 gr/kg/day.

14. The composition of claim 1, wherein the pharmaceutical composition is adapted for administration via parenteral, intravenous, oral, intramuscular, intraaortal, intrahepatic, intragastric, intranasal, intrapulmonary, intraperitoneal, subcutaneous, rectal, vaginal, intraosseal or dermal delivery.

15. The composition of claim 1, wherein the pharmaceutical composition is in powder, tablet, gelatin, gelcap, capsule, soft-gel, chewable or liquid form.

16. The composition of claim 1, further comprise one or more vitamins, minerals, amino acids, lipids, nucleic acids, co-factors, pro-vitamins, and combinations of mixtures thereof.

17. A composition for modulating a cardiac hypertrophy comprising an amount of a Calmodulin kinase kinase modulator sufficient to change the activity of the Calmodulin kinase kinase.

18. A pharmaceutical composition for treating a cardiac hypertrophy comprising a pharmaceutically effective amount of a Calmodulin kinase kinase inhibitor sufficient to treat a patient suspected of having cardiac hypertrophy.

19. The composition of claim 18, wherein the composition increases the amount of intracellular Calmodulin kinase kinase, intracellular Calmodulin kinase kinase mRNA, the stability of intracellular Calmodulin kinase kinase mRNA and combinations thereof.

20. The composition of claim 18, wherein the composition decreases the amount of intracellular Calmodulin kinase kinase, intracellular Calmodulin kinase kinase mRNA, the stability of intracellular Calmodulin kinase kinase mRNA and combinations thereof.

21. The composition of claim 18, wherein the composition increases the kinase activity of the Calmodulin kinase kinase.

22. The composition of claim 18, wherein the composition decreases the kinase activity of the Calmodulin kinase kinase mRNA.

23. A method of treating a modulating muscle mass comprising:
administering to a patient in need thereof a composition comprising a Calmodulin kinase kinase inhibitor in a pharmaceutically acceptable carrier, in an amount insufficient to treat the cardiac hypertrophy.

24. The method of claim 23, wherein the muscle is cardiac muscle.

25. A method for treating or preventing hypertrophic cardiomyopathy in a mammal, the method comprising administering a Calmodulin kinase kinase inhibitor to the mammal, wherein the Calmodulin kinase kinase inhibitor is administered in an amount effective to treat or prevent heart failure in the mammal.

26. The method of claim 25, wherein the mammal is a human.

27. The method of claim 25, wherein the hypertrophic cardiomyopathy results from hypertension; ischemic heart disease; exposure to a cardiotoxic compound; myocarditis; thyroid disease; viral infection; gingivitis; drug abuse; alcohol abuse; periocarditis; atherosclerosis; vascular disease; hypertrophic cardiomyopathy; acute myocardial infarction; left ventricular systolic dysfunction; coronary bypass surgery; starvation; an eating disorder; or a genetic defect.

28. The method of claim 25, wherein the Calmodulin kinase kinase inhibitor is administered prior to, during, after the onset of cardiac hypertrophy.

29. The method of claim 25, wherein the Calmodulin kinase kinase inhibitor is administered prior to, during, after the diagnosis of heart failure in the mammal.

30. The method of claim 25, wherein the Calmodulin kinase kinase inhibitor is administered prior to, during, after compensatory cardiac hypertrophy.

31. A method of treating a disease in a mammal resulting from deficiencies of cardiac output comprising administering to the mammal a therapeutically effective amount of a pharmaceutical composition comprising a Calmodulin kinase kinase inhibitor.

32. A method of modulating muscle mass comprising administering to a patient in need of an increase or a decrease in muscle mass an amount of a Calmodulin kinase kinase modulator sufficient to alter the muscle mass.

33. The method of claim 32, wherein the muscle is selected from cardiac and skeletal.

34. The method of claim 32, wherein the patient has muscle weakness and reduced pulmonary function, wherein the Calmodulin kinase kinase modulator increased muscle output.

35. The method of claim 32, wherein the muscle weakness is caused by acute muscle usage.

36. The method of claim 32, wherein the muscle weakness is chronic.

37. A method for diagnosing hypertrophic cardiomyopathy in a mammal, the method comprising measuring the Calmodulin kinase kinase activity from the mammal suspected of having the hypertrophic cardiomyopathy to the levels of kinase activity in a mammal known to have a normal cardiac function.

38. A method for screening compounds for the ability to prevent or treat the manifestations of heart failure, comprising:
contacting a Calmodulin kinase kinase to one or more candidate substances; and
measuring the effect of the candidate substance on the kinase activity of the Calmodulin kinase kinase, wherein a candidate substance identified thereby is subsequently tested for improvement in the physiologic function of the heart of the mouse, thereby identifying a compound as therapeutic.

39. The method of claim 38, wherein a control Calmodulin kinase kinase is a Val₂₆₉ to Leu₂₆₉ mutant, wherein known inhibitors of Calmodulin kinase kinase do not affect Calmodulin kinase kinase Val₂₆₉ to Leu₂₆₉ kinase activity.

40. The method of claim 38, wherein the candidate substance is a derivative of a naphthoyl fused benzimidazole compound.

41. The method of claim 38, wherein the candidate substance is a derivative of a STO-609.

42. The method of claim 38, wherein the candidate substance is derived from the Calmodulin kinase kinase-specific inhibitor STO-609.

43. A method for ameliorating the effects of physical exertion, the method comprising the administration to a person in need of such amelioration a composition of claim 1.

44. A diet for supporting a patient with cardiac hypertrophy comprising a nutritionally effective amount of a Calmodulin kinase kinase modulator to reduce the symptoms associated with cardiac hypertrophy and decreases muscle mass.

45. The diet of claim 44, further comprising essential fats of between about 0.1 to 10% total Kcal/day; carbohydrates restricted to about 0.1 to 10% total Kcal/day; and a protein content of between about 0.1 to 10% total Kcal/day of the diet.

46. The diet of claim 44, wherein the Calmodulin kinase kinase modulator is provided in a beverage concentrate comprising: one or more carbohydrates, one or more electrolytes and one or more Calmodulin kinase kinase modulators in a concentration of between about 0.1% to about 10.0% weight percent.

47. The beverage of claim 46, further defined as comprising the following ingredients:

| Ingredient | Approximate Concentration |
|---|---|
| Potassium | 2 meq/l |
| Sodium | 26 meq/l |
| Glucose | 4% |
| Pyruvate | 1% |
| a Calmodulin kinase kinase modulator | 0.1 to 10% |
| Emulsifier | 0.1 to 2.0% |
| water | balance. |

48. The beverage of claim 46, wherein the Calmodulin kinase kinase modulator comprises a Calmodulin kinase kinase inhibitor.

49. A food composition comprising:
a mixture of ingredients selected to make one or more snacks, soups, salads, cakes, cookies, crackers, breads, ice creams, yogurts, puddings, custards, baby foods, medicinal foods, sports bars, breakfast cereals and beverages; and
a Calmodulin kinase kinase inhibitor comprising a concentration of between about 0.5% to about 5.0% of the composition.

50. The composition of claim 49, wherein the wherein the food composition comprises supplemental a dietary fiber selected from the group consisting of apple fiber, corn bran, soy fiber, pectin, guar gum, gum ghatti, and gum arabic, as well as mixtures thereof.

51. The composition of claim 49, wherein the wherein the food composition comprises a binder material selected from the group consisting of rice flour, wheat flour, oat flour, corn flour, rye flour and potato flour, as well as mixtures thereof.

52. A nutritional supplement comprising a nutritionally effective amount of a Calmodulin kinase kinase modulator sufficient to modulate muscle size.

53. A transgenic mouse displaying manifestations of cardiac hypertrophy selected from the group consisting of: shortness of breath, angina, palpitations, lightheadedness, fatigue, syncope, dyspnea, elevated pressure in the left ventricle and left atrium and combinations thereof relative to a control mouse, wherein the genome of the mouse comprises a promoter operably linked to a nucleotide sequence encoding a Calmodulin kinase kinase, and a Calmodulin kinase kinase in its cardiac tissue at a level that is at least 3-fold higher than in cardiac tissue of a control mouse.

54. The transgenic mouse of claim 53, wherein the operable linked promoter is an inducible promoter, a cardiac-specific promoter or a murine α-myosin heavy chain gene promoter.

55. The transgenic mouse of claim 53, wherein the Calmodulin kinase kinase is selected from a CaM-KKα a CaM-KKβ and combinations thereof.

56. The transgenic mouse of claim 53, wherein the Calmodulin kinase kinase is a Val₂₆₉ to Leu₂₆₉ mutant.

57. A method for producing a transgenic mouse expressing a Calmodulin kinase kinase mRNA in cardiac tissue, the method comprising:
introducing into an embryonal cell of a mouse a cardiac-specific gene promoter operably linked to a nucleotide sequence encoding a Calmodulin kinase kinase protein, wherein the promoter is capable of directing the expression of the nucleotide sequence encoding a Calmodulin kinase kinase protein in a cardiac-specific manner;
transplanting the transgenic embryonal target cell formed thereby into a recipient female parent; and
identifying at least one transgenic offspring containing the nucleotide sequence in the offspring's genome, wherein at from 8 to 18 months of age the offspring displays manifestations of cardiac hypertrophy selected from the group consisting of: shortness of breath, angina, palpitations, lightheadedness, fatigue, syncope, dyspnea, elevated pressure in the left ventricle and left atrium and combinations thereof relative to a control mouse, and expresses Calmodulin kinase kinase protein mRNA in its cardiac tissue at a level which is at least 3-fold higher than in cardiac tissue of a control offspring.

58. The method of claim 57, wherein the offspring is further characterized by not overexpressing the Calmodulin kinase kinase protein mRNA in skeletal muscle.

59. The method of claim 57, wherein the Calmodulin kinase kinase protein is selected from the group consisting of CaM-KKα a CaM-KKβ and combinations thereof.

60. A method for screening compounds for the ability to prevent or treat the manifestations of heart failure in a mouse, comprising:
providing a transgenic mouse from 8 to 18 months of age displaying manifestations of cardiac hypertrophy selected from the group consisting of: shortness of breath, angina, palpitations, lightheadedness, fatigue, syncope, dyspnea, elevated pressure in the left ventricle and left atrium and combinations thereof relative to a control mouse, wherein the genome of the mouse comprises an α-myosin heavy chain gene promoter operably linked to a nucleotide sequence encoding Calmodulin kinase kinase mRNA that expresses Calmodulin kinase kinase mRNA in its cardiac tissue at a level that is at least 3-fold higher than in cardiac tissue of a control mouse;
administering a compound to the mouse; and
measuring an improvement in the physiologic function of the heart of the mouse and thereby identifying a compound as therapeutic.


SEQUENCE LISTING (12 sequences)

SEQ ID NO: 1 - DNA, 23 bp, Artificial
  Synthetic Oligonucleotide
ctgtgactgagctgcctggtgac

SEQ ID NO: 2 - DNA, 21 bp, Artificial
  Synthetic Oligonucleotide
ggtggctatgatgaagggaat

SEQ ID NO: 3 - DNA, 20 bp, Artificial
  Synthetic Oligonucleotide
tgccggtagaagatgaggtc

SEQ ID NO: 4 - DNA, 20 bp, Artificial
  Synthetic Oligonucleotide
agccctcagtttgcttttca

SEQ ID NO: 5 - DNA, 20 bp, Artificial
  Syntheic Oligonucleotide
gacgggctgaggttgtttta

SEQ ID NO: 6 - DNA, 20 bp, Artificial
  Synthetic Oligonucleotide
ttgtgctggaagataagaaa

SEQ ID NO: 7 - DNA, 20 bp, Artificial
  Synthetic Oligonucleotide
ctcccaaggagagacgactg

SEQ ID NO: 8 - DNA, 20 bp, Artificial
  Synthetic Oligonucleotide
cccttggtgacgtactcgtt

SEQ ID NO: 9 - DNA, 20 bp, Artificial
  Synthetic Oligonucleotide
tgcccatttatgagggctac

SEQ ID NO: 10 - DNA, 20 bp, Artificial
  Synthetic Oligonucleotide
ggcatacaggtccttcctga

SEQ ID NO: 11 - DNA, 24 bp, Artificial
  Synthetic Oligonucleotide
gtgtgaacggatttggccgtatgg

SEQ ID NO: 12 - DNA, 24 bp, Artificial
  Synthetic Oligonucleotide
tcatacttggcaggtttctccagg